SEQUENCE LISTING (78 sequences)

SEQ ID NO: 1 - DNA, 33 bp, Varicella zoster
  misc_feature (7)..(12): PvuI restriction enzyme cleavage site
agagagcagctgcgtataacgaatccggtcaga

SEQ ID NO: 2 - DNA, 32 bp, Varicella zoster
  misc_feature (7)..(14): NotI restriction enzyme cleavage site
agagaggcggccgctcgtagaagtggtgacgt

SEQ ID NO: 3 - protein, 549 aa, Varicella zoster
  MISC_FEATURE (3)..(9): FLAG tag
  MISC_FEATURE (10)..(16): FLAG tag
  MISC_FEATURE (17)..(23): FLAG tag
MSDYKDHDGDYKDHDIDYKDDDDKAMAQVPAPNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRAAAGS

SEQ ID NO: 4 - DNA, 22 bp, Homo sapiens
caggtgcagctgcaggagtccg

SEQ ID NO: 5 - DNA, 22 bp, Homo sapiens
caggtgcagctgcaggagtcgg

SEQ ID NO: 6 - DNA, 21 bp, Homo sapiens
caggtacagctgcagcagtca

SEQ ID NO: 7 - DNA, 22 bp, Homo sapiens
caggtgcagctacagcagtggg

SEQ ID NO: 8 - DNA, 21 bp, Homo sapiens
  misc_feature (1)..(21): K = G or T
  misc_feature (1)..(21): W = A or T
  misc_feature (1)..(21): Y = C or T
gaggtgcagctgktggagwcy

SEQ ID NO: 9 - DNA, 23 bp, Homo sapiens
  misc_feature (1)..(23): K = G or T
  misc_feature (1)..(23): R = A or G
caggtccagctkgtrcagtctgg

SEQ ID NO: 10 - DNA, 22 bp, Homo sapiens
cagatcaccttgaaggagtctg

SEQ ID NO: 11 - DNA, 22 bp, Homo sapiens
caggtcaccttgaaggagtctg

SEQ ID NO: 12 - DNA, 23 bp, Homo sapiens
  misc_feature (1)..(23): S = C or G
  misc_feature (1)..(23): R = A or G
caggtgcagctggtgsartctgg

SEQ ID NO: 13 - DNA, 23 bp, Homo sapiens
  misc_feature (1)..(23): S = C or G
  misc_feature (1)..(23): B = C, G or T
cagtctgtsbtgacgcagccgcc

SEQ ID NO: 14 - DNA, 22 bp, Homo sapiens
  misc_feature (1)..(22): W = A or T
tcctatgwgctgacwcagccac

SEQ ID NO: 15 - DNA, 23 bp, Homo sapiens
  misc_feature (1)..(23): Y = C or T
  misc_feature (1)..(23): R = A or G
tcctatgagctgayrcagcyacc

SEQ ID NO: 16 - DNA, 20 bp, Homo sapiens
  misc_feature (1)..(20): R = A or G
  misc_feature (1)..(20): Y = C or T
cagcctgtgctgactcaryc

SEQ ID NO: 17 - DNA, 23 bp, Homo sapiens
  misc_feature (1)..(23): D = A, G or T
  misc_feature (1)..(23): Y = C or T
cagdctgtggtgacycaggagcc

SEQ ID NO: 18 - DNA, 23 bp, Homo sapiens
  misc_feature (1)..(23): W = A or T
  misc_feature (1)..(23): M = A or C
  misc_feature (1)..(23): K = G or T
cagccwgkgctgactcagccmcc

SEQ ID NO: 19 - DNA, 23 bp, Homo sapiens
  misc_feature (1)..(23): S = C or G
tcctctgagctgastcaggascc

SEQ ID NO: 20 - DNA, 21 bp, Homo sapiens
  misc_feature (1)..(21): Y = C or T
cagtctgyyctgaytcagcct

SEQ ID NO: 21 - DNA, 22 bp, Homo sapiens
aattttatgctgactcagcccc

SEQ ID NO: 22 - DNA, 23 bp, Homo sapiens
  misc_feature (1)..(23): R = A or G
  misc_feature (1)..(23): D = A, G or T
gacatccrgdtgacccagtctcc

SEQ ID NO: 23 - DNA, 23 bp, Homo sapiens
  misc_feature (1)..(23): W = A or T
  misc_feature (1)..(23): R = A or G
gaaattgtrwtgacrcagtctcc

SEQ ID NO: 24 - DNA, 23 bp, Homo sapiens
gatattgtgmtgacbcagwctcc

SEQ ID NO: 25 - DNA, 22 bp, Homo sapiens
gaaacgacactcacgcagtctc

SEQ ID NO: 26 - DNA, 22 bp, Homo sapiens
tgaggagacagtgaccagggtg

SEQ ID NO: 27 - DNA, 22 bp, Homo sapiens
tgaggagacggtgaccagggtg

SEQ ID NO: 28 - DNA, 22 bp, Homo sapiens
tgaggagacggtgaccagggtt

SEQ ID NO: 29 - DNA, 21 bp, Homo sapiens
tgaagagacggtgaccattgt

SEQ ID NO: 30 - DNA, 23 bp, Homo sapiens
tgaggagacggtgaccgtggtcc

SEQ ID NO: 31 - DNA, 20 bp, Homo sapiens
ggttggggcggatgcactcc

SEQ ID NO: 32 - DNA, 21 bp, Homo sapiens
  misc_feature (1)..(21): S = C or G
  misc_feature (1)..(21): R = A or G
sgatgggcccttggtggargc

SEQ ID NO: 33 - DNA, 20 bp, Homo sapiens
  misc_feature (1)..(20): S = C or G
taggacggtsascttggtcc

SEQ ID NO: 34 - DNA, 20 bp, Homo sapiens
gaggacggtcagctgggtgc

SEQ ID NO: 35 - DNA, 20 bp, Homo sapiens
tttgatttccaccttggtcc

SEQ ID NO: 36 - DNA, 20 bp, Homo sapiens
tttgatctccaccttggtcc

SEQ ID NO: 37 - DNA, 20 bp, Homo sapiens
tttgatctccagcttggtcc

SEQ ID NO: 38 - DNA, 20 bp, Homo sapiens
tttgatatccactttggtcc

SEQ ID NO: 39 - DNA, 20 bp, Homo sapiens
tttaatctccagtcgtgtcc

SEQ ID NO: 40 - DNA, 21 bp, Homo sapiens
agaccttggggctggtcgggg

SEQ ID NO: 41 - DNA, 21 bp, Homo sapiens
gaggctcagcgggaagacctt

SEQ ID NO: 42 - DNA, 21 bp, Homo sapiens
gaggtggcattggagggaatg

SEQ ID NO: 43 - DNA, 21 bp, Homo sapiens
gacggaatgggctcgtgtgga

SEQ ID NO: 44 - DNA, 21 bp, Homo sapiens
cacatccggagccttggtggg

SEQ ID NO: 45 - DNA, 36 bp, Homo sapiens
  misc_feature (7)..(14): NcoI restriction enzyme cleavage site
agagagccatggcccaggtgcagctgcaggagtccg

SEQ ID NO: 46 - DNA, 36 bp, Homo sapiens
  misc_feature (7)..(14): NcoI restriction enzyme cleavage site
agagagccatggcccaggtgcagctgcaggagtcgg

SEQ ID NO: 47 - DNA, 35 bp, Homo sapiens
  misc_feature (7)..(14): NcoI restriction enzyme cleavage site
agagagccatggcccaggtacagctgcagcagtca

SEQ ID NO: 48 - DNA, 36 bp, Homo sapiens
  misc_feature (7)..(14): NcoI restriction enzyme cleavage site
agagagccatggcccaggtgcagctacagcagtggg

SEQ ID NO: 49 - DNA, 35 bp, Homo sapiens
  misc_feature (1)..(35): W = A or T
  misc_feature (1)..(35): Y = C or T
  misc_feature (1)..(35): K = G or T
  misc_feature (7)..(14): NcoI restriction enzyme cleavage site
agagagccatggccgaggtgcagctgktggagwcy

SEQ ID NO: 50 - DNA, 37 bp, Homo sapiens
  misc_feature (1)..(37): K = G or T
  misc_feature (1)..(37): R = A or G
  misc_feature (7)..(14): NcoI restriction enzyme cleavage site
agagagccatggcccaggtccagctkgtrcagtctgg

SEQ ID NO: 51 - DNA, 36 bp, Homo sapiens
  misc_feature (7)..(14): NcoI restriction enzyme cleavage site
agagagccatggcccagatcaccttgaaggagtctg

SEQ ID NO: 52 - DNA, 36 bp, Homo sapiens
  misc_feature (7)..(14): NcoI restriction enzyme cleavage site
agagagccatggcccaggtcaccttgaaggagtctg

SEQ ID NO: 53 - DNA, 37 bp, Homo sapiens
  misc_feature (1)..(37): S = C or G
  misc_feature (1)..(37): R = A or G
  misc_feature (7)..(14): NcoI restriction enzyme cleavage site
agagagccatggcccaggtgcagctggtgsartctgg

SEQ ID NO: 54 - DNA, 36 bp, Homo sapiens
  misc_feature (1)..(36): S = C or G
  misc_feature (1)..(36): B = C, G or T
  misc_feature (7)..(13): MluI restriction enzyme cleavage site
agagagacgcgtacagtctgtsbtgacgcagccgcc

SEQ ID NO: 55 - DNA, 35 bp, Homo sapiens
  misc_feature (1)..(35): W = A or T
  misc_feature (7)..(13): MluI restriction enzyme cleavage site
agagagacgcgtatcctatgwgctgacwcagccac

SEQ ID NO: 56 - DNA, 36 bp, Homo sapiens
  misc_feature (1)..(36): B = C, G or T
  misc_feature (1)..(36): Y = C or T
  misc_feature (7)..(13): MluI restriction enzyme cleavage site
agagagacgcgtatcctatgagctgayrcagcyacc

SEQ ID NO: 57 - DNA, 33 bp, Homo sapiens
  misc_feature (1)..(33): R = A or G
  misc_feature (1)..(33): Y = C or T
  misc_feature (7)..(13): MluI restriction enzyme cleavage site
agagagacgcgtacagcctgtgctgactcaryc

SEQ ID NO: 58 - DNA, 36 bp, Homo sapiens
  misc_feature (1)..(36): D = A, G or T
  misc_feature (1)..(36): Y = C or T
  misc_feature (7)..(13): MluI restriction enzyme cleavage site
agagagacgcgtacagdctgtggtgacycaggagcc

SEQ ID NO: 59 - DNA, 36 bp, Homo sapiens
  misc_feature (1)..(36): W = A or T
  misc_feature (1)..(36): K = G or T
  misc_feature (1)..(36): M = A or C
  misc_feature (7)..(13): MluI restriction enzyme cleavage site
agagagacgcgtacagccwgkgctgactcagccmcc

SEQ ID NO: 60 - DNA, 36 bp, Homo sapiens
  misc_feature (1)..(36): S = C or G
  misc_feature (7)..(13): MluI restriction enzyme cleavage site
agagagacgcgtatcctctgagctgastcaggascc

SEQ ID NO: 61 - DNA, 34 bp, Homo sapiens
  misc_feature (1)..(34): Y = C or T
  misc_feature (7)..(13): MluI restriction enzyme cleavage site
agagagacgcgtacagtctgyyctgaytcagcct

SEQ ID NO: 62 - DNA, 35 bp, Homo sapiens
  misc_feature (7)..(13): MluI restriction enzyme cleavage site
agagagacgcgtaaattttatgctgactcagcccc

SEQ ID NO: 63 - DNA, 36 bp, Homo sapiens
  misc_feature (1)..(36): R = A or G
  misc_feature (1)..(36): D = A, G or T
  misc_feature (7)..(13): MluI restriction enzyme cleavage site
agagagacgcgtagacatccrgdtgacccagtctcc

SEQ ID NO: 64 - DNA, 36 bp, Homo sapiens
  misc_feature (1)..(36): R = A or G
  misc_feature (1)..(36): W = A or T
  misc_feature (7)..(13): MluI restriction enzyme cleavage site
agagagacgcgtagaaattgtrwtgacrcagtctcc

SEQ ID NO: 65 - DNA, 36 bp, Homo sapiens
  misc_feature (1)..(36): M = A or C
  misc_feature (1)..(36): B = C, G or T
  misc_feature (1)..(36): W = A or T
  misc_feature (7)..(13): MluI restriction enzyme cleavage site
agagagacgcgtagatattgtgmtgacbcagwctcc

SEQ ID NO: 66 - DNA, 35 bp, Homo sapiens
  misc_feature (7)..(13): MluI restriction enzyme cleavage site
agagagacgcgtagaaacgacactcacgcagtctc

SEQ ID NO: 67 - DNA, 34 bp, Homo sapiens
  misc_feature (7)..(12): HindIII restriction enzyme cleavage site
agagagaagctttgaggagacagtgaccagggtg

SEQ ID NO: 68 - DNA, 34 bp, Homo sapiens
  misc_feature (7)..(12): HindIII restriction enzyme cleavage site
agagagaagctttgaggagacggtgaccagggtg

SEQ ID NO: 69 - DNA, 34 bp, Homo sapiens
  misc_feature (7)..(12): HindIII restriction enzyme cleavage site
agagagaagctttgaggagacggtgaccagggtt

SEQ ID NO: 70 - DNA, 33 bp, Homo sapiens
  misc_feature (7)..(12): HindIII restriction enzyme cleavage site
agagagaagctttgaagagacggtgaccattgt

SEQ ID NO: 71 - DNA, 35 bp, Homo sapiens
  misc_feature (7)..(12): HindIII restriction enzyme cleavage site
agagagaagctttgaggagacggtgaccgtggtcc

SEQ ID NO: 72 - DNA, 34 bp, Homo sapiens
  misc_feature (1)..(34): S = C or G
  misc_feature (7)..(14): NotI restriction enzyme cleavage site
agagaggcggccgctaggacggtsascttggtcc

SEQ ID NO: 73 - DNA, 34 bp, Homo sapiens
  misc_feature (7)..(14): NotI restriction enzyme cleavage site
agagaggcggccgcgaggacggtcagctgggtgc

SEQ ID NO: 74 - DNA, 34 bp, Homo sapiens
  misc_feature (7)..(14): NotI restriction enzyme cleavage site
agagaggcggccgctttgatttccaccttggtcc

SEQ ID NO: 75 - DNA, 34 bp, Homo sapiens
  misc_feature (7)..(14): NotI restriction enzyme cleavage site
agagaggcggccgctttgatctccaccttggtcc

SEQ ID NO: 76 - DNA, 34 bp, Homo sapiens
  misc_feature (7)..(14): NotI restriction enzyme cleavage site
agagaggcggccgctttgatctccagcttggtcc

SEQ ID NO: 77 - DNA, 34 bp, Homo sapiens
  misc_feature (7)..(14): NotI restriction enzyme cleavage site
agagaggcggccgctttgatatccactttggtcc

SEQ ID NO: 78 - DNA, 34 bp, Homo sapiens
  misc_feature (7)..(14): NotI restriction enzyme cleavage site
agagaggcggccgctttaatctccagtcgtgtcc

(19) United States
(12) Patent Application Publication
(10) Pub. No.: US 2007/0072240 A1
(43) Pub. Date: Mar. 29, 2007

(54) Method for producing an in vitro expression library

(75) Inventors: Ole Henrik Brekke, Oslo (NO); John Stacy, Oslo (NO); Louise Kausmally, Oslo (NO)

Correspondence Address: ROTHWELL, FIGG, ERNST & MANBECK, P.C., 1425 K STREET, N.W., SUITE 800, WASHINGTON, DC, 20005, US

(73) Assignee: Affitech AS, Oslo (NO)

(21) Appl. No.: 11/522,978

(22) Filed: Sep. 19, 2006

Related U.S. Application Data
(63) Continuation of application No. 10/379,996, filed on Mar. 6, 2003, now abandoned.

(30) Foreign Application Priority Data
May 14, 2002 (GB) 0211015.3
Nov. 29, 2002 (GB) 0227977.6

(51) Int. Cl. C40B 30/06; C40B 40/10; G01N 33/53; A61K 39/00

(52) U.S. Cl. 435/7.1; 424/184.1; 514/44

(57) ABSTRACT

The present invention relates to an antibody expression library derived from a patient which has been immunochallenged with one or more foreign antigens associated with a particular disease or foreign agent, wherein said patients have been immunochallenged with the foreign antigens at a time point such that they still contain a repertoire of antibody producing cells which are enriched with cells producing antibodies directed to said foreign antigens associated with said disease or foreign agent, or at a time point such that they are still in an active phase of immune response to said foreign antigens associated with said disease or foreign agent. Methods of producing such expression libraries are also disclosed.

This application is a continuation of U.S. Ser. No. 10/379,996, filed Mar. 6, 2003, which claims priority from United Kingdom Application 0211015.3, filed May 14, 2002 and United Kingdom Application 0227977.6, filed Nov. 29, 2002. These prior applications are incorporated herein by reference.

This invention relates to antibody expression libraries derived from immunochallenged patients and methods of preparing such libraries. The invention further relates to the use of these expression libraries to screen and isolate high affinity and highly diverse recombinant human antibodies exhibiting desired properties and which may be suitable for use as therapeutic antibodies or for in vivo diagnosis. Novel expression vectors for use in the production of expression libraries are also described.

Molecular libraries are important tools in many areas of molecular and cellular biology and in the identification and development of new drugs and therapeutic and diagnostic agents. Such libraries generally contain genetic material, for example fragments of genes or nucleic acid sequences which may in turn be associated with a suitable vehicle which allows the fragments to be amplified and manipulated, e.g. a plasmid. Such libraries may be screened at the nucleic acid level, i.e. by screening for the desired sequence or sequences using a nucleotide probe. Alternatively such libraries can be designed so that the polypeptides encoded by the nucleic acid fragments or genes are expressed and screening can be carried out using an appropriate “target” molecule to which it is desired that the selected polypeptide should bind. The latter type of libraries where the encoded peptides are capable of being expressed are known as “expression libraries”.

In the development of therapeutic antibodies (and to a large extent diagnostic antibodies) several success criteria must be met. The antibodies must show high specificity, the antibodies should have high affinity for the relevant target molecule and the antibodies should express well in different expression systems. Another important aspect is that one should be able to isolate a pool of antibodies showing specificity against different epitopes on the same antigen, i.e the repertoire of isolated human antibodies should be as broad as possible so as to be able to identify antibodies against the most effective, neutralizing epitope(s).

All in vitro antibody selection systems, e.g. phage or ribosomal display technologies, demand the selection of antibody fragments within an unnatural context. Either the antibody fragments are fused to a phage protein (in phage display) or as a fusion to ribosomes (in ribosomal display). In addition, these selection systems demand several rounds of selection to reduce the number of antibody candidates to be screened. This may drive the selection pressure towards only a few antibody candidates that behave well in their unnatural context and which are reactive against one or only a few epitopes.

Although improved in vitro selection methods have been developed which increase the throughput in such selection assays, thereby decreasing the number of rounds of selection required, the inherent problems associated with the selection of antibody fragments in an unnatural context and the biased repertoire associated with this selection remain.

There is therefore a need for alternative improved in vitro selection systems wherein the number of rounds of selection can be reduced or even eliminated, wherein the antibodies can be selected in a more natural context and wherein the repertoire from which the antibodies are selected is as broad or diverse as possible so as to identify antibodies reactive against the most effective epitopes.

Surprisingly, it has now been found that these needs can be met by deriving and screening in vitro antibody expression libraries from post-disease or immunochallenged patients. These expression libraries contain high affinity and highly diverse human antibodies which can be directly generated and screened as expression libraries and do not need to be subjected to many rounds of selection in an unnatural context. Advantageously, libraries can be generated and be ready for screening very quickly, e.g. in up to 4 weeks. In addition, a throughput of 10000 to 40000 screened clones a month is feasible from such patient libraries.

The present invention is based on the idea that individuals that have been exposed to a particular disease or foreign antigen or agent and especially an infectious disease, hold the genetic information for the expression of extremely good antibodies against the particular disease antigen(s) or foreign antigen(s) in question. Advantageously it has been found that populations of antibody producing cells from such patients are enriched for cells which make antibodies directed to the disease antigen or foreign antigen in question. Such antibodies are highly diverse in their nature and reactive to a large number of epitopes displayed on the particular foreign antigen(s) associated with the disease or foreign agent.

In contrast to expression libraries generated from a healthy naive donor which has not been exposed to a particular disease or foreign agent or antigen, or even from a healthy donor which has been exposed to a particular disease or foreign agent or antigen in the past but recovered from that disease, foreign agent or antigen some time ago, e.g. at least a few months ago, e.g. at least two or three months ago, or more than a year ago, expression libraries generated from the antibody producing cells from patients which have more recently been or are being immunochallenged with a particular disease or foreign antigen or foreign agent, have a repertoire of antibodies which is smaller in terms of the number of antigens recognised but which is enriched and more diverse for the antibodies of interest. The repertoires of antibodies generated from healthy naive donors which have not been exposed to a particular disease, foreign agent or antigen, or from healthy donors which had recovered from a particular disease, foreign agent or antigen some time ago, e.g. in the time frames discussed above, would be so large that it would not be possible to directly screen such repertoires as an expression library. Disadvantageously, such repertoires would therefore have to undergo some form of selection before screening. Thus, it can be seen that the selection of an appropriate population of antibody producing cells from appropriate patients which have come into contact with particular foreign antigens, agents or diseases in accordance with the present invention gives rise to significant advantages in terms of the repertoire of antibodies contained in the expression libraries and the fact that direct screening without prior selection steps can be carried out.

Thus, the repertoire of antibodies contained in an expression library generated in accordance with the present invention from patients which have been exposed to a particular disease, foreign antigen or foreign agent, will be different from the repertoire produced in expression libraries derived from a naive donor or from healthy donors which had recovered from a particular disease, foreign agent or antigen some time ago, e.g. in the time frames discussed above.

Moreover, the antibody producing cells from such non-naive patients or non-healthy patients selected in accordance with the present invention, will generally produce the high affinity antibodies (e.g. IgG antibodies) associated with an immune response as opposed to the lower affinity antibodies (IgM antibodies) which are associated with non-stimulated antibody producing cells, or the primary immune response. Thus, the antibodies identified and isolated from these non-naive or non-healthy donor expression libraries in accordance with the present invention by screening are generally of high affinity and more appropriate for use as therapeutic antibodies or antibodies for in vivo diagnosis.

Thus, the present invention provides an antibody expression library derived or obtainable from a patient which has been immunochallenged with one or more foreign antigens associated with a particular disease or foreign agent.

The term “high affinity” as used herein in connection with antibodies, generally refers to antibodies or antibody fragments with affinities for antigen in the range of 10⁻⁸ M to 10⁻¹⁰ M or higher.

“patient” as used herein, for example a “non-naive patient” or a “non-healthy patient”, refers to an individual which is undergoing some kind of immunochallenge with one or more foreign antigens associated with a particular disease or foreign agent. Such patients are for example in an active phase of an immune response or for example contain a repertoire of antibody producing cells which are enriched with cells producing antibodies directed to the foreign antigen or antigens associated with the particular disease or foreign agent in question. Such patients will generally show the symptoms of such an immunochallenge, e.g. show the symptoms of disease or will have been deliberately immunochallenged, e.g. in the case of administration of a vaccine. Thus, naive donors, i.e. donors which have not been exposed to the foreign antigen in question or healthy donors which have been exposed to a particular disease or foreign agent or antigen in the past but recovered from that disease, foreign agent or antigen some time ago, e.g. at least a few months ago, e.g. at least two or three months ago, or more than a year ago, are excluded, as they will not be undergoing such an immunochallenge. Thus, the patients from which the expression libraries of the present invention are derived have been exposed to or immunochallenged with the foreign antigen or agent relatively recently, e.g. not more than two months ago.

“immunochallenged” as used herein in connection with patients refers to patients which have been exposed in some way to one or more foreign antigens which are associated with a particular disease or foreign agent. The exposure to foreign antigen can occur in any appropriate way and includes exposure to foreign antigens associated with a particular disease or foreign agent by way of the patient contracting the disease, by for example the patient being infected with particular disease associated bacteria, virus, fungi, etc., or by the patient in some other way coming into contact with a foreign agent, e.g. by the patient coming into contact with an allergen. This exposure also includes deliberate exposure of a patient to one or more particular foreign antigens associated with a particular disease or foreign agent, e.g. by vaccination protocols.

The exposure may be a first exposure to the foreign antigen(s) in question or may be a second or subsequent exposure to the antigen(s), e.g. may be a re-infection with a particular disease or infectious agent, may be a re-exposure to a particular foreign agent, e.g. an allergen, or may be a second or further “booster” exposure to a particular vaccine. The exposure may also be a first and continued exposure to a foreign antigen e.g. in the case of exposure to an autoimmune disease.

In preferred embodiments of the invention, the immunochallenge will be a second or subsequent exposure to particular foreign antigen(s).

Patients which have recovered from an exposure to a particular foreign antigen (e.g. have recovered from a particular disease or infectious agent or have dealt with or eliminated a foreign agent such as an allergen) and have been re-exposed/re-immunochallenged with the foreign antigen in question are particularly preferred as sources for the expression libraries of the invention, as their repertoire of antibody producing cells will include cells which produce antibodies which are effective in combatting the foreign antigen/disease/agent in question, i.e. their repertoire of antibodies will contain particularly efficacious antibodies. The antibodies which have combatted the disease or foreign agent should be the best candidates for neutralisation of the foreign antigen(s) in question. Such antibodies should show at least the correct specificity and probably also a broad range of binding properties to a given antigen, e.g. have the ability to bind different epitopes.

The main requirement for the patients from which the antibody expression libraries of the present invention are derived is that they have been immunochallenged/exposed to the disease, foreign agent or foreign antigen(s) at a time point such that they still contain a repertoire of antibody producing cells which are enriched with cells producing antibodies directed to the foreign antigen or antigens associated with the particular disease or foreign agent in question.

Appropriate time points may vary depending on the type of immunochallenge in question. For example, in the case of an immunochallenge by a foreign antigen which is eventually significantly reduced or eliminated from a patient by the immune system, e.g. the type of immunochallenge presented by most diseases, infectious agents or foreign agents such as allergens, such patients will have been immunochallenged/exposed at a time point sufficiently recently such that they still contain a repertoire of antibody producing cells which are enriched with cells producing antibodies directed to the foreign antigen or antigens associated with the particular infectious or foreign agent in question. In the case of an immunochallenge by a foreign antigen which is not significantly reduced or eliminated from a patient, e.g. by a “self” antigen associated with an autoimmune disease, the expression libraries can be derived from patients at any time point such that they still contain a repertoire of antibody producing cells which are enriched with cells producing antibodies directed to the foreign antigen or antigens (or “self” antigens if applicable) associated with the particular disease or foreign agent in question. In the latter case it can be seen that the time window in which the antibody producing cells can be isolated in order to derive the expression libraries of the invention is increased.

“enriched” as used herein in connection with populations or repertoires of antibody producing cells in a patient refers to a population of antibody producing cells which contains a significantly increased number of antibody producing cells which produce antibodies specifically reactive with the foreign antigen(s) in question compared to the number of antibody producing cells producing antibodies specifically reactive with the foreign antigen(s) in question in a population of antibody producing cells obtained from a naive donor which has not been exposed to the foreign antigen(s) in question, or a healthy donor which has been exposed to a particular disease or foreign agent or antigen in the past but recovered from that disease, foreign agent or antigen some time ago, e.g. at least a few months ago, e.g. at least two or three months ago, or more than a year ago. A preferred comparison will be with a naive donor. Such enriched populations may for example contain at least 0.1 specific B cells (antibody producing cells) per thousand non-specific B cells (antibody producing cells).

Methods of determining the statistical significance of differences in parameters such as the number of antibody producing cells between two or more samples are well known and documented in the art and any such method can be used. Generally, whichever test is chosen to determine significance levels, a probability value of <0.05 is desired in order for the difference to be regarded as significant.

Such an enrichment of antibody producing cells will occur as part of the natural immune response to foreign antigen, as antibody producing cells (B cells/B lymphocytes) which are capable of making antibodies directed to the foreign antigen in question will be stimulated to proliferate and will therefore increase in number. Such an enrichment will occur over time and reach a maximum. However, generally the enrichment will naturally tail off once the amount of antigen in question is reduced or eliminated as there will be no further antigen induced stimulation of B cell proliferation and the existing B cells will be removed by naturally occurring biological mechanisms, e.g. by cell death. The enrichment will however sometimes be maintained, for example in cases where the foreign antigen is not significantly eliminated, e.g. in immunochallenges with autoimmune diseases. In such cases, the time window in which the antibody producing cells can be isolated in order to derive the expression libraries of the invention is increased.

Put another way, the patients from which the antibody expression libraries of the present invention are derived have been immunochallenged/exposed to the disease, foreign agent or foreign antigen(s) at a time point such that they are still in an active phase of immune response to the foreign antigen, etc., in question. Patients in an active phase of immune response can readily be identified by a person skilled in the art. For example, such patients will be actively producing specific antibodies in response to foreign antigen. Thus, for example the presence of a high serum titre of specific antibodies to the foreign antigen in question is indicative of such appropriate patients. Preferably this high serum titre of specific antibodies will be combined with a relatively low serum titre of non-specific antibodies, thereby evidencing the enrichment of the antibody producing cells. Again the serum titres of candidate patients can be compared to the serum titres of naive donors or healthy donors as described above in order to assess whether or not the serum titre of antibodies to a particular foreign antigen is significantly higher in the candidate patients.

Thus, there is a finite time window after exposure to antigen in which the B cells from the patient which are used to provide the genetic material for the antibody expression libraries of the invention need to be isolated in order to obtain the benefits of the enriched B-cell population.

The length of time after exposure to antigen to meet this requirement may vary from patient to patient, may depend on the disease, foreign agent or foreign antigen in question (e.g. whether or not the disease, etc., is an infectious disease or is otherwise associated with a foreign antigen which is eventually significantly reduced or eliminated by the patient, or is associated with a foreign antigen which is not significantly eliminated, e.g. an autoimmune disease, as discussed above), the source of the B cells in the patient (e.g. circulating B cells as opposed to e.g. B cells in the lymphoid tissues) and also on whether or not a primary, secondary or further response to the foreign antigen is being mounted. However, the suitability of a patient can readily be determined if desired, by taking a sample of antibody producing cells (B-cells) from the patient, e.g. by taking a blood sample, and carrying out a standard in vitro assay (e.g. an ELISA assay or ELISPOT assay, Czerkinsky et al., 1983, J. Immunol. Methods, vol 65:109-121) using the relevant foreign antigen as a target antigen and measuring the degree of immunoreaction. Preferably the degree of immunoreaction with a control antigen is also assessed in order to provide an indication of the level of enrichment of the sample for the desired antibodies. A low or relatively low degree of immunoreaction with a control antigen is evidence that the expression libraries derived from these patients will contain fewer irrelevant antibodies, i.e. will be enriched and diverse for antibodies against the antigen in question.

This immunoreaction measurement can be done one or more times to monitor the progress and degree of a patient's immune response to an antigen and assess (by for example an appropriate comparison with a naive donor) whether or not a suitably enriched population of B cells is present. In this way the optimum time to harvest antibody producing cells (which contain the genetic material from which the expression library will be derived) from a patient can be identified. In addition, patients which are not appropriate or are no longer appropriate to provide material for expression library generation can readily be identified.

A preferred assay in this regard is the ELISPOT assay mentioned above. Such an assay is especially suitable for testing circulating B cells and is based on the coating of a surface with the particular foreign antigen to which it is desired to obtain antibodies (and by which the patient is being immunochallenged) and adding a defined number of B cells. B cells secreting antibodies that bind to the antigen can be detected by conventional ELISA detection. This assay only detects B cells which are secreting specific antibodies and not B cells with specific membrane bound antibodies, so the actual number of B cells with specific antibodies may actually be higher than the test suggests.

Preferably however the immunochallenged patients will have been exposed to the foreign antigen(s) relatively recently before the isolation of the antibody producing cells from which the expression libraries are derived, e.g. the patients will have received a first or subsequent exposure to a particular foreign antigen(s) or foreign agent, or the disease will have broken out up to 40 or 30 days before isolation, more preferably up to 20 or 10 days before isolation, most preferably 4 to 10 or 15, or 6 to 7 days before isolation of the antibody producing cells. In other words, the antibody expression libraries are preferably derived from material from a patient obtained up to 40 days (e.g. 30 to 40 days), more preferably up to 30 days, 20 days or 15 days (e.g. 20 to 30 days or 10 to 15 or 20 days), most preferably up to 10 days or 7 days (e.g. 4 to 10 days or 6 to 7 days), or approximately one week after a first or subsequent exposure to a particular foreign antigen(s) or foreign agent or the outbreak of disease.

“Outbreak of disease” as used herein generally refers to the first visible sign or symptom of the disease in question, for example the appearance of spots or lesions. Such signs and symptoms will vary from disease to disease depending on the foreign antigen in question, but will be well known and easily recognised for a particular disease by a person skilled in the art.

Viewed alternatively, a further preferred time point at which to isolate the antibody producing cells from an immunochallenged patient is a short time prior to the maximum serum levels of the specific antibodies to the foreign antigen(s) in question being reached, e.g. a time point up to approximately one week before, e.g. 1 to 10 days before the patient shows the highest serum levels of antibodies (antibody producing cells) against the particular foreign antigen(s) in question. This time point generally occurs about one week, e.g. 4 to 10 or 15, or 6 to 7 days after immunochallenge, e.g. approximately one week or two weeks after a first or subsequent exposure to a particular foreign antigen(s) or foreign agent or the outbreak of disease. Thus, a most preferred time point at which to isolate the antibody producing cells from an immunochallenged patient is 6 to 7 days before the patient shows the highest serum levels of antibodies (antibody producing cells) against the particular foreign antigen(s) in question.

Again this time point can readily be identified/assessed for a patient by standard assays, e.g. ELISA or ELISPOT assays as described above. In general, samples of antibody producing cells will be obtained from patients at regular and appropriate time points and the appropriate sample from which the expression library should be derived determined by way of the ELISPOT or ELISA assays as mentioned above. As discussed in more detail below, cell samples can readily be stored until the appropriate sample from which the expression library should be derived is determined.

The selection of an appropriate patient as a source of antibody producing cells from which to derive the expression libraries of the invention may also depend on the type of antibodies it is desired to have in the repertoire. For example, if it is desired to generate a library comprising an enriched IgM repertoire, then the B cells will preferably be isolated after a first exposure of a patient to the foreign antigen, agent, disease, etc. On the other hand, if it is desired that the repertoire reflects an enriched pool of antibodies in the IgG format, which is the preferred format, or another format such as IgA, IgD or IgE, the B cells may be isolated after a first exposure to the foreign antigen, etc., but more preferably are isolated after a second or subsequent exposure.

In the case of IgE antibodies, it would be particularly desirable to isolate a hypersensitive IgE repertoire from patients which are mounting a response to a particular allergen. Antibodies identified from such expression libraries in accordance with the present invention could be used as anti-allergenic antibody fragments, competing with the IgE response and thus inhibiting an allergic reaction.

Where the exposure to foreign antigen occurs via the contraction of a naturally occurring disease, these patients may also be referred to herein as “post-disease” patients.

A further aspect of the invention provides a method for producing an in vitro antibody expression library from a patient which has been immunochallenged with one or more foreign antigens associated with a particular disease or foreign agent, said method comprising the steps of:

a) isolating one or more populations of antibody producing cells from said patient, or otherwise obtaining said populations, and

b) cloning nucleic acid fragments comprising sequences encoding antibody variable domains, or fragments thereof, from said antibody producing cells into an appropriate expression vector to form a library of said expression vectors capable of expressing a library of antibody molecules.

Appropriate immunochallenged patients from which the antibody producing cells are isolated in step a) are as defined above.

Once the library of expression vectors has been generated, the encoded antibody molecules can then be expressed in an appropriate expression system and screened using appropriate techniques which are well known and documented in the art. Thus the above defined method of the invention may comprise the further steps of c) expressing the library of expression vectors in an appropriate expression system and optionally d) screening the expressed library for antibodies with desired properties.

Antibody expression libraries produced by the methods of the invention and antibodies selected or identified therefrom form yet further aspects of the invention. Thus, the present invention further provides antibody expression libraries obtainable by the methods of the invention and antibodies selected or identified therefrom.

“Antibody expression library” as used herein can refer to a collection of molecules (i.e. two or more molecules) at either the nucleic acid or protein level. Thus, this term can refer to a collection of expression vectors which encode a plurality of antibody molecules (i.e. at the nucleic acid level) or can refer to a collection of antibody molecules after they have been expressed in an appropriate expression system (i.e. at the protein level). Alternatively the expression vectors/expression library may be contained in suitable host cells in which they can be expressed. The antibody molecules which are encoded or expressed in the expression libraries of the invention can be in any appropriate format, e.g. may be whole antibody molecules or may be antibody fragments, e.g. single chain antibodies (e.g. scFv antibodies), Fv antibodies, Fab antibodies, Fab′2 fragments, diabodies, etc.

Antibody molecules identified by, derived from, selected from or obtainable from the antibody expression libraries of the invention form a yet further aspect of the invention. Again these antibody molecules may be proteins or nucleic acids encoding antibody molecules, which nucleic acids may in turn be incorporated into an appropriate expression vector and/or be contained in a suitable host cell.

Indeed where the term “antibody molecule” is used herein, this should be taken to include the antibody molecule per se, but also includes nucleic acid molecules encoding said antibody molecules, or expression vectors containing said nucleic acid molecules.

The patients from which the antibody expression libraries are derived or produced may have been immunochallenged/exposed to any disease which is associated with one or more foreign antigens. Preferred diseases are infectious diseases caused by infectious agents such as Varicella Zoster Virus (VZV), HIV, CMV, Hepatitis (and in particular Hepatitis B or C), Herpes, Meningococcus (and in particular Meningococcus A, B or C) and group A Streptococcus. Particularly preferred diseases/immunochallenges are those caused by Meningococcus B and especially preferably the immunochallenge is stimulated by meningococcus B Outer membrane vesicles (OMV), e.g. from Neisseria meningitidis, e.g. by the vaccine 44/76 developed at the Norwegian Institute of Public Health (Fredriksen et al., 1991, NIPH Ann., vol 14:67-80) or the vaccine NZ98/254 developed from the New Zealand strain NZ98/254. Other particularly preferred diseases/immunochallenges are those caused by VZV infection.

Other preferred diseases/immunochallenges are cancers (for example pancreatic cancers, lymphomas, sarcomas or melanomas), autoimmune diseases (for example Coeliac disease, Lupus or Rheumatoid arthritis) or allergic diseases. Thus, it can be seen that “foreign antigens” as used herein in accordance with the present invention includes antigens which are normally self antigens but which are recognised as foreign antigens by the particular patient in question from which the antibody expression library is derived.

The population(s) of antibody producing cells (B cells) isolated from a patient in accordance with the present invention can be derived from one or more appropriate sources, for example from one or more of peripheral blood, cells from bone marrow, spleen cells, tonsils or any other secondary lymphoid tissue, tumour infiltrating lymphocytes, tissues or organs affected by an autoimmune disease, or from any other tissues or fluids or other samples known to harbour antibody producing B cells. In some cases, the appropriate sources of B cells will depend on the disease or immunochallenge to which antibodies are sought. For example, in a preferred embodiment of the invention where antibodies to particular cancers are sought, a preferred source of B cells would be the tumour infiltrating lymphocytes which are targeting the tumour in question. In a further preferred embodiment of the invention where antibodies effective against particular autoimmune diseases are sought, a preferred source of B cells would be antibody producing cells which are associated with or which are targeting the tissue or organ which is effected by the autoimmune disease in question. The appropriate tissues in this regard would be readily determined from the literature. For example, it is known that Rheumatoid arthritis effects the joints and that Coeliac disease effects the gut. Thus, these tissues may be preferred sources of B cells where these autoimmune diseases were concerned.

The derivation of B cells from a tissue source or other cellular source which is effected by the disease in question and to which an immune response is being directed is particularly advantageous as the repertoire of antibodies produced by this population should be further enriched for B cells which produce the desired antibodies. Examples of this is deriving the B-cell population from the tumour infiltrating lymphocytes associated with a particular cancer or deriving the B-cell population from the tissue or organ affected by a particular autoimmune disease as described above.

The total B-lymphocyte pool from one or more of the selected sources can be isolated from the patient and used to generate antibody expression libraries in accordance with the invention. As explained above, because of the selection criteria used for the immunochallenged patients from which the B cells are isolated, these B cell populations are already enriched for B cells making antibodies to the foreign antigen or antigens in question. However, in further preferred embodiments of the invention the lymphocyte pool may be further enriched for the desired lymphocytes before the expression library is made. Thus, in a yet further embodiment of the invention, step a) of the method is followed by one or more steps wherein the isolated or obtained population of antibody producing cells is enriched for antibody producing cells which produce the desired antibody molecules. Antibody expression libraries produced or obtainable by such methods and antibodies selected or identified therefrom form yet further aspects of the invention.

The use of methods wherein the isolated population of antibody producing cells is further enriched for antibody producing cells which produce the desired antibody molecules are particularly advantageous if it is desired to isolate antibodies from the expression library which maintain the original variable heavy and variable light chain pairing from the patient. This is sometimes desirable as clearly it is likely that the very presence of a particular original pairing of light and heavy chains in the B cell population derived from an immunochallenged patient means that this particular combination of heavy and light chains is likely to be functional in recognising antigen. When antibody expression libraries are created (as will be described in more detail below) the heavy chain genes and light chain genes, or portions thereof, are generally cloned separately and brought back together by inserting them into the appropriate position in an appropriately designed expression vector. In such systems the heavy and light chains are thus partnered together in an essentially random manner meaning that the original combination will be a relatively rare occurrence. Thus, methods whereby the original pairing can be retained are sometimes desirable. Where the isolated population of B cells is further enriched, the enriched population will contain only a few subsets of B cells expressing different antibodies. These sub-populations therefore contain only a few naturally occurring combinations of variable heavy (VH) and variable light (VL) genes. Thus, when the light and heavy chains are cloned from the enriched subpopulation into expression vectors in the normal-way, the probability that the VL and VH pairing will correspond to a naturally occurring combination is significantly increased.

Any appropriate method of further enrichment may be used. A preferred method however involves stimulating the initial isolated population of B cells in vitro with the specific antigen or antigens to which it is desired that the antibodies be directed. Contacting the B cells with such antigens will activate and stimulate the selective proliferation of the B cells in the population which recognise the antigen, thereby further enriching the population of B cells with B cells which recognise the antigen and therefore contain genes encoding potential candidate antibodies against the particular antigen. Methods of stimulation of B cells with antigen in vitro in this way would be well known to the person skilled in the art.

A further method of enrichment is to use a solid support, e.g. magnetic beads, coated with specific antigen, to isolate B cells with antigen specific membrane bound antibodies from the initial population of B cells obtained from the patient.

Alternatively the population of B cells can be enriched by dilution and cultivation to reduce the size of the repertoire. Thus, in the expression libraries of the present invention one of the ways of retaining the original combination of heavy and light chains is by diluting the original isolated B-cell repertoire from the patient so as to generate several small sub-populations of B cells which contain only a few subsets of B cells expressing different antibodies. Preferably such sub-populations contain B cells expressing only one or two different antibody molecules. These sub-populations (which will generally need to be expanded after dilution in order to obtain sufficient B cells to work with by for example inducing the cells to proliferate) therefore contain only a few naturally occurring combinations of variable heavy (VH) and variable light (VL) genes. Thus, when the light and heavy chains are cloned from the diluted subpopulation into expression vectors in the normal way, the probability that the VL and VH pairing will correspond to a naturally occurring combination is significantly increased.

Once the enriched B lymphocyte pool has been generated using one or more of the above described methods or any other appropriate method, the nucleic acids encoding variable heavy and variable light chains, or fragments thereof, contained within the various B lymphocytes are cloned into appropriate expression vectors by standard techniques.

It should be noted that in the methods and expression libraries of the invention, once appropriate patients from which a population of antibody producing cells can be isolated has been identified and the appropriate population of said cells have been isolated at an appropriate time and optionally further enriched as described above, the antibody expression libraries need not be generated immediately, providing the genetic material contained in the cells can be kept intact thereby enabling the library to be made at a later date. Thus, for example the cells, a cell lysate, or nucleic acid, e.g. RNA or DNA derived therefrom, can be stored until a later date by appropriate methods, e.g. by freezing, and the expression libraries generated at a later date when desired.

Any appropriate expression system can be used to express the antibody libraries of the invention and the expression vectors into which the antibody variable domains are cloned are selected accordingly. Preferred expression systems are bacterial or other prokaryotic expression systems and most preferably E. coli expression systems. Such bacterial expression systems are especially preferred because these allow the members of the antibody expression library to be expressed in a more natural context. Other expression systems, e.g. phage display, covalent display (WO98/37186, Fitzgerald, K., 2000, Drug Discovery Today, vol 5:253-258) or ribosomal display systems can also be used to express the antibody libraries (and again appropriate expression vectors can be selected accordingly). One of the advantages with expressing the antibody expression libraries of the present invention in any system, is the fact that direct expression can take place (i.e. without the many rounds of panning and selection usually required to reduce the number of candidate antibodies) due to the reduction in size of the total repertoire of candidate antibodies contained in the libraries and the enrichment of the repertoire for the antibodies of interest by the appropriate selection of patients to be used as the source of antibody molecules. However, as mentioned above, systems such as phage, covalent or ribosomal display do suffer the disadvantage that the antibodies are expressed and selected in an unnatural context.

The cloning of the various nucleic acid fragments comprising sequences encoding antibody variable domains or fragments thereof from the heavy and light chain antibody genes into appropriate expression vectors can be carried out using conventional genetic engineering techniques which are well known to a person in the art and described in the literature.

An exemplary general method is described below, although it will be appreciated that steps of this method may be varied or omitted as appropriate.

Total RNA from the isolated B-lymphocyte population (or the further enriched population) is extracted by appropriate methods which are standard and conventional in the art. cDNA is then synthesised from the RNA by appropriate methods, e.g. using random hexamer oligonucleotides. Again these are processes known to persons skilled in the art.

As indicated above, the populations of B-lymphocytes isolated from immunochallenged patients as defined herein will be distinct from populations isolated from a non-immunochallenged/naive patient. Thus, such isolated B lymphocyte populations (or the further enriched populations) provide a yet further aspect of the invention. Further aspects provided are libraries of isolated nucleic acid molecules derived from such populations of B-lymphocytes (or the further enriched populations), e.g. a library of RNA or cDNA molecules derived from such B-lymphocytes. These libraries of isolated nucleic acid molecules may optionally be cloned into expression vectors to form expression libraries.

The cDNA pool is then subjected to a primary PCR reaction with oligonucleotides that hybridise to the IgG constant region of the heavy chain of antibody genes and oligonucleotides that hybridise to the 5′ end of the variable heavy chain region of antibody genes. A PCR reaction is also set up for the amplification of the variable light (VL) chain pool of kappa and lambda classes. Such oligonucleotides may be designed based on known and publicly available immunoglobulin gene sequence database information.

It is important to note that the above described PCR reactions are set up to clone only the antibodies in the IgG form. These are preferred as they are generally associated with a more mature immune response and generally exhibit higher affinity than IgM antibodies, thereby making them more desirable for certain therapeutic and diagnostic applications. Clearly however oligonucleotides can be designed which will allow the cloning of one or more of the other forms of immunoglobulin molecules, e.g. IgM, IgA, IgE and IgD if desired or appropriate.

The primary PCR products are then subjected to a secondary PCR reaction with new oligonucleotide sets that hybridise to the 5′ and 3′ ends of the antibody variable domains V-Heavy, V-light kappa and V-light lambda (as appropriate depending on whether the primary PCR reaction with which the new oligonucleotide sets are used was designed to amplify portions of the heavy or light chain antibody genes). These oligonucleotides advantageously include DNA sequences specific for a defined set of restriction enzymes (i.e. restriction enzyme sites) for subsequent cloning. The selected restriction enzymes must be selected so as not to cut within human antibody V-gene segments. Such oligonucleotides may be designed based on known and publicly available immunoglobulin gene sequence and restriction enzyme database information. However, preferred restriction enzyme sites to be included are NcoI, Hind III, MluI and NotI. The products of such secondary PCR reactions are repertoires of various V-heavy, V-light kappa and V-light lambda antibody fragments/domains. This type of secondary PCR reaction is therefore generally carried out when the expression library format of interest is a scFv or Fv format, wherein only the VH and VL domains of an antibody are present.

Libraries of such repertoires of cloned fragments comprising the variable heavy chain regions, or fragments thereof, and/or variable light chain regions, or fragments thereof, of antibody genes derived from the B lymphocytes of immunochallenged patients as defined herein form further aspects of the invention. These libraries comprising cloned variable regions may optionally be inserted into expression vectors to form expression libraries.

Alternatively, if desired, the primary and secondary PCR reactions can be set up so as to retain all or part of the constant regions of the various heavy and/or light antibody chains contained in the isolated B cell population. This is desirable when the expression library format is a Fab format, wherein the heavy chain component comprises VH and CH domains and the light chain component comprises VL and CL domains. Again libraries of such cloned fragments comprising all or part of the constant regions of heavy and/or light antibody chains form further aspects of the invention.

Once they have been cloned, the nucleic acid molecules encoding the various portions of antibody molecules, e.g. the heavy chains or light chains of antibodies or portions thereof, e.g. VH and/or VL chains, may be further diversified using standard techniques, for example by mutation involving the addition, deletion and/or substitution of one or more nucleotides in a controlled (e.g. site directed mutagenesis) or random manner, or by domain swapping, cassette mutagenesis, chain shuffling etc. Synthetic nucleotides may be used in the generation of the diverse nucleic acid sequences. Thus, all or part of the nucleic acids encoding the antibody domains can be synthesised chemically.

Preferably however the isolated nucleic acid molecules encoding the various antibody domains for making up the expression library are not subject to further diversification at this stage and correspond to the sequences as found in vivo and which are likely to be functional in antigen binding due to the fact that they are expressed by B lymphocytes in a patient in response to a specific immunochallenge.

The appropriate variable gene fragments may then be cloned into an expression vector so as to generate an expression library of scFv fragments. The structure and organisation of vectors to produce scFv antibody fragments are known in the art. In general, to generate such scFv fragments nucleic acid fragments encoding variable light chain and variable heavy chain domains are cloned into an expression vector in a single reading frame where the variable heavy and variable light chains are linked by a nucleic acid encoding a peptide linker. The expression vector is preferably an E coli expression vector and is advantageously also designed so that the expressed antibody fragments contain a detection tag such as a MYC tag.

It will be appreciated however from the discussion above that the methods and expression libraries of the invention are not limited to antibodies in the single chain format and other formats can equally be generated, for example Fab fragments, Fab′2 fragments, Fv fragments, diabodies, etc., in accordance with methods which are well known in the art. In addition other types of expression vector can be used. In particular other forms of prokaryotic expression vectors can be used, as well as different types of display vectors such as phage, covalent or ribosomal display vectors. E. coli vectors are however preferred for the various reasons outlined herein.

The main requirement of an expression vector is that it contains all the necessary components required for obtaining expression of the appropriate nucleic acid molecule encoding the polypeptide of interest in the particular expression system chosen. Thus, the expression vectors, as well as the nucleic acid fragments encoding the antibody molecules, may optionally additionally contain other appropriate components, for example origins of replication, inducible promoters for initiating transcription and protein expression, antibiotic resistance genes and markers, general tags, detection tags such as myc tags or reporter molecules, primer binding sites to enable amplification of the constructs by e.g. PCR, or any other desirable sequence elements. Appropriate sources and positioning of such additional components within the library constructs so that they perform their desired function would be well within the normal practice of a skilled person in the art.

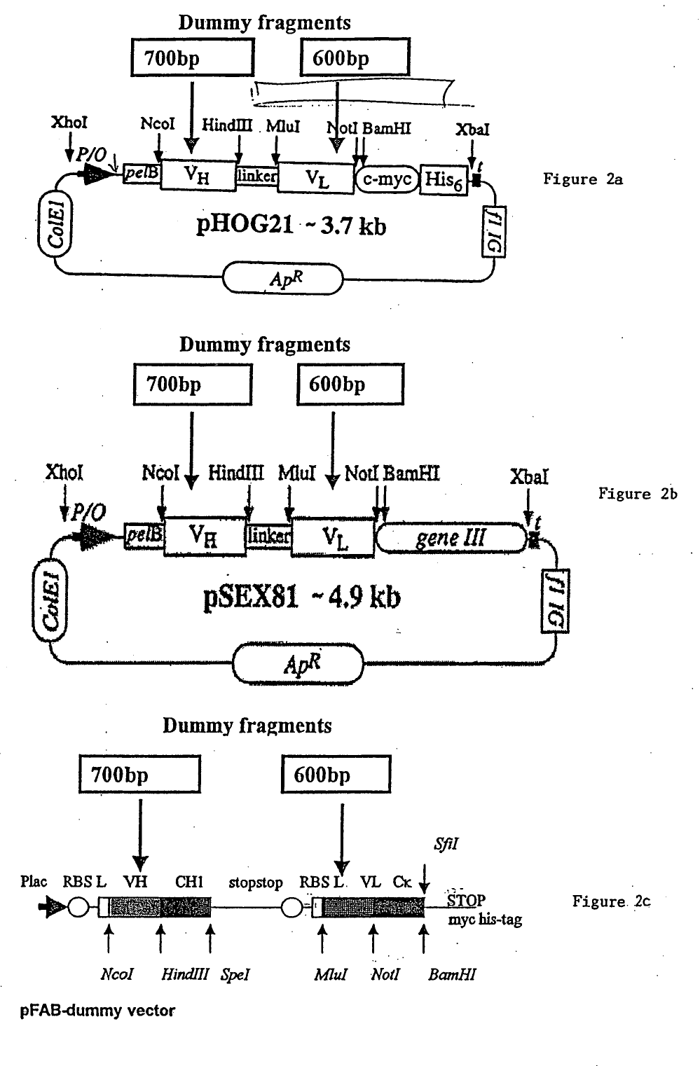

Preferred expression vectors for use in the invention are the pHOG vector and the pSEX vector, the general structures of which are shown in FIG. 2. The pHOG vector is suitable for expression of soluble scFv fragments in bacterial systems such as E. Coli. The structure of the pHOG vector shown in FIG. 2 can readily be adapted for the expression of a different format of antibody, e.g. a Fab antibody. The pSEX vector is a phagemid vector which is suitable for expressing scFv antibody fragments in phage display systems. In such vectors the scFv fragments are expressed as a fusion protein with gpIII, a filamentous bacteriophage coat protein or a fusion with some other appropriate bacteriophage coat protein or fragment thereof. Again such vectors can readily be adapted to express other formats of antibody, e.g. Fab fragments.

After cloning into appropriate expression vectors, the antibody expression library is transformed into E. coli cells (or other appropriate host cells depending on the vector system used) such as XL-1 blue. Transformation can be carried out by electroporation or any other appropriate method, after which the library is plated onto nutrient (e.g. agar) plates. Generally the transformed E. coli (or other host) are also plated with relevant antibiotics, e.g. ampicillin, so that only the host cells containing the expression vector with a resistance gene can grow. Generally the host cells are also plated with transcription inhibitors to inhibit protein expression.

The particular plating technique will differ depending on the technique chosen for screening the colonies. The aim here is generally to choose a technique with the ability to screen as many candidates as possible in order to isolate and detect the colonies in the library that produce the best antibody candidates. There are several techniques which achieve this aim and which are well known and described in the art. Any of these techniques may be used. However, a preferred method is the filter screening method summarized below (and also described in Example 1) which can be used to screen approximately 40,000 different colonies a week.

This method can of course be readily adapted depending on the expression system chosen. However, for an E. coli expression system, the bacterial colonies containing plasmids encoding scFv fragments (or other format of antibody) are picked by a colony picking robot and are inoculated to generate master plates of single colony bacterial stocks.

The picked colonies are gridded on a nitrocellulose membrane and grown overnight on non-inducing medium (i.e. a medium which does not allow protein expression).

The nitrocellulose membrane with bacterial colonies are induced to express scFv fragments by adding an appropriate inducer and are brought into contact with a secondary nitrocellulose membrane coated with the specific antigen to which it is desired to identify reactive antibodies. The appropriate inducer used will depend on the design of the expression vector. However a preferred inducer is IPTG which induces expression from the LAC promoter.

The antigen coated membranes are subjected to washing steps and specific detection of bound scFv antibodies is carried out by incubating with appropriate reagents. The appropriate reagents will again depend on the design of the expression system. However, if for example the scFv fragments also contain a myc tag then incubation with anti-MYC specific antibodies followed by detection of such antibodies, for example with HRP-conjugated anti mouse antibodies is appropriate. The membrane is developed by ECL (or any other appropriate method).

By comparing developed membranes with or without specific antigens, specific spots can be identified which correspond to a location in the stock master plate which express a potential antibody candidate. These candidates can then be further analysed by returning to the stock master plate.

Thus, the antibody expression library is generally screened for antibody molecules which interact with a particular target antigen, e.g. a foreign antigen associated with a particular disease or foreign agent. Once one or more antibody molecules have been identified using the methods of the invention these molecules (or the nucleic acid encoding them) can be isolated and purified.

Thus, a further aspect of the present invention provides a method of identifying and/or isolating one or more antibody molecules exhibiting desired properties from an antibody expression library as defined herein, said method comprising the step of screening an antibody expression library of the invention for molecules which display certain properties.

A preferred aspect of the invention thus provides a method of identifying and/or isolating from an antibody expression library as defined herein one or more antibody molecules which is a specific binding partner for a target antigen, said method comprising the steps of a) screening an expression library of the invention for antibody molecules which bind to a particular target antigen and b) identifying and/or isolating the relevant library member.

Once appropriate nucleic acid fragments encoding candidate antibody molecules with particular properties have been identified, the gene pool encoding candidate polypeptides can, if desired, be subjected to affinity maturation, for example to try and identify candidate antibodies with further improved properties. Such affinity maturation can be performed by carrying out any conventional form of mutagenesis, including but not limited to the addition, deletion and/or substitution of one or more nucleotides in a controlled (e.g. site directed mutagenesis) or random manner, error-prone PCR, domain swapping, cassette mutagenesis and chain shuffling, etc., prior to repetition of the screening cycle.

When one or more antibody molecule candidates have been selected, identified and/or purified using the methods and expression libraries of the invention, these candidates, or a component, fragment, variant, or derivative thereof may be manufactured and if desired formulated with at least one pharmaceutically acceptable carrier or excipient. Such manufactured antibody molecules, or components, fragments, variants, or derivatives thereof, are also encompassed by the present invention. Alternatively, these antibody molecules may take the form of nucleic acids encoding antibody molecules, which nucleic acids may in turn be incorporated into an appropriate expression vector and/or be contained in a suitable host cell. Thus, nucleic acid molecules encoding said antibody molecules, or expression vectors containing said nucleic acid molecules form further aspects of the invention.

Once a particular antibody molecule, or a component, fragment, variant, or derivative thereof, has been selected, identified, etc., in accordance with the present invention, the expression vector encoding the selected antibody can readily be used (or adapted for use) to produce sufficient quantities of the antibody molecule by expression in appropriate host cells or systems and isolating the antibody molecules from the host cell or system or from the growth medium or supernatant thereof, as appropriate. Alternatively, said antibody molecules may be produced by other appropriate methods, e.g. by chemical synthesis of the nucleic acid encoding the antibody and expression in a suitable host or in an in vitro transcription system.

Thus, a yet further aspect of the invention provides a method of manufacturing a specific antibody molecule comprising the steps of identifying a specific antibody molecule which is a binding partner for a target antigen according to the methods of the invention as described above, manufacturing said identified antibody molecule, or a component, fragment, variant, or derivative thereof and optionally formulating said manufactured antibody molecule with at least one pharmaceutically acceptable carrier or excipient. Antibody molecules (or components, fragments, variants, or derivatives thereof), identified, manufactured or formulated in this way form yet further aspects of the invention.

Said variants or derivatives of an antibody molecule include peptoid equivalents, molecules with a non-peptidic synthetic backbone and polypeptides related to or derived from the original identified antibody molecule polypeptide wherein the amino acid sequence has been modified by single or multiple amino acid substitutions, additions and/or deletions which may alternatively or additionally include the substitution with or addition of amino acids which have been chemically modified, e.g. by deglycosylation or glycosylation. Conveniently, such derivatives or variants may have at least 60, 70, 80, 90, 95 or 99% sequence identity to the original polypeptide from which they are derived.

Said variants or derivatives further include the conversion of one format of antibody molecule into another format (e.g. conversion from Fab to scFv or vice versa), or the conversion of an antibody molecule to a particular class of antibody molecule (e.g. the conversion of an antibody molecule to IgG or a subclass thereof, e.g. IgG1 or IgG3, which are particularly suitable for therapeutic antibodies). Said variants or derivatives further include the association of antibody molecules with further functional components which may for example be useful in the downstream applications of said antibodies. For example the antibody molecules may be associated with components which target the antibodies to a particular site in the body, detectable moieties useful for example in imaging or other diagnostic applications, or peptides which mimic the Fc effector functions of the constant regions of antibody molecules (e.g. by activating complement or binding to Fc receptors), by for example making pepbody molecules such as those described by Affitech AS (see PCT/GB01/05301).

Clearly, the main requirement for such components, fragments, variants, or derivative antibody molecules is that they retain their original functional activity in terms of ability to bind a specific antigen or have improved functional activity.

The antibody expression libraries of the invention can be screened against more than one target antigen. For example, the libraries of the invention can be screened against similar targets either to avoid or to obtain cross-reactive antibodies. For example in the course of generating human antibodies against infectious diseases the libraries can be screened against different strains of the disease causing agent. Antibodies specific for one strain must recognise a disease specific antigen. Conversely, antibodies binding different strains must recognise common antigens among the strains. At least the antibodies must recognise common or structurally similar epitopes on antigens. Such antibodies, identified by screening the libraries of the invention with two or more different but related target antigens, e.g. target antigens from different strains of a particular infectious agent (i.e. antibodies identified by differential screening) are particularly good candidates for use as therapeutic or prophylactic antibodies against a specific strain or different strains of a disease causing agent and form a preferred embodiment of the invention.

Antibodies which have the ability to recognise more than one strain of an infectious agent clearly have advantages in terms of more widespread use. The expression libraries of the invention can readily be used to identify such antibodies as they can be used for differential screening against a number of targets. Indeed, experiments show that the libraries of the present invention are useful to detect a significant number of cross-reactive antibodies. For example, Example 2 describes how an expression library derived from patients challenged with a vaccine derived from a particular strain of Neisseria meningitidis B (Norwegian strain 44/76) and screened with the OMV target antigen from such a strain can also be screened for antibodies which recognise the OMV target antigen from a second strain of Neisseria meningitidis B (New Zealand strain NZ-98/254), and cross-reactive antibodies (i.e. antibodies which recognise both target antigens) identified. In this example approximately 10% cross-reactive clones were detected.

Thus, the use of the expression libraries of the invention for differential screening with different target antigens to identify cross-reactive antibodies forms a yet further aspect of the invention.

The libraries of the present invention can also be used in the development of new and improved vaccines. Usually vaccines are composed of attenuated virus or bacteria or composite fractions of bacteria such as capsules or membrane vesicles. Common for most vaccines are that the detailed compositions and the parts of the vaccine that are most immunogenic with respect to inducing a protective immune response are often not known, so efforts to discover good immunogens are important for the development of safe and effective vaccines.

The tapping of the antibody repertoire from vaccinated individuals as described herein can be used to provide information on which immunogens foster the most effective and protective antibodies. The utilisation of a vaccinee's antibody repertoire as a tool to discover or isolate such immunogens has been proposed and termed “Reverse vaccinology” (Burton D R, Nature Reviews in Immunology 2, 706-713 (2002)). The patient antibody libraries and in particular the vaccinee antibody libraries produced in accordance with the present invention can readily be used for such reverse vaccinology, which generally involves the identification of antibodies which can interact with a particular vaccine and then identifying the antigen/epitope of the vaccine with which these antibodies are interacting (i.e. identifying the immunogen). Once the immunogen has been identified at the protein level, this can be used to deduce the nucleic acid sequence encoding the immunogen and, if desired, the native gene encoding the immunogen can be isolated by standard methods. This will then allow the production of pure recombinant vaccines using an appropriate expression system. Such vaccines are likely to be more safe and effective than conventional vaccines and may comprise one or more different immunogens.

Thus, a yet further embodiment of the invention provides the use of the libraries of the invention to develop vaccines or in reverse vaccinology to identify the immunogens of a vaccine which stimulate the most effective and protective antibodies.

The invention further provides a method to develop and/or produce a recombinant vaccine comprising the steps of:

a) screening an antibody library of the invention derived from a vaccinee, against one or more vaccines;

b) identifying the immunogen(s) of the vaccine to which the antibodies recognising the vaccine become bound;

c) deducing the nucleic acid sequence encoding one or more of the identified immunogens;

d) optionally isolating the native gene(s) encoding the immunogen(s);

e) using said nucleic acid sequence or native gene to produce a recombinant vaccine.

The screening step (a) is generally carried out against the vaccine used to vaccinate the patient from which the antibody library is derived, but is preferably also carried out against one or more related vaccines in order that the immunogens identified in step (b) are more likely to be common to more than one vaccine and therefore have a wider ranging use as a recombinant vaccine. For example the libraries are preferably not only screened against the particular strain of infectious agent with which the patient was vaccinated but are also screened against one or more related strains. Thus the screening step (a) preferably involves differential screening as described above.

The step (b) of identifying the immunogens can be carried out by any appropriate methods which would be well known to those skilled in the art of protein characterisation. Generally the positive antibodies binding the vaccine are isolated and the immunogen of the vaccine identified using for example methods such as western blot, immuno-histochemistry, native gel electrophoresis, detection of protein spots on two-dimensional gel electrophoresis, isolation of protein followed by N-terminal sequencing, or affinity purification of the immunogen from the vaccine. The amino acid sequence can be used to deduce the nucleic acid sequence and the native gene encoding the immunogen can be isolated by for example sequence homology search and PCR amplification of the deduced gene. The nucleic acid sequence or isolate of the gene encoding the immunogen can be expressed in a suitable expression system so as to generate a pure recombinant vaccine.

Once the recombinant vaccines have been developed these can be formulated in an appropriate way for administration to patients. Such formulation and appropriate modes and regimens of administration would be well known to a person skilled in the art. Such recombinant vaccines form a yet further embodiment of the invention.

Successful use of the methods and expression libraries of the invention is dependent on high levels of expression of the correct antibody polypeptide by a particular clone. The inventors have developed a novel type of expression vector which helps to improve this by virtue of only allowing in frame antibody fragments to be detectably expressed. Such expression vectors form a yet further preferred aspect of the invention.

Thus, a yet further aspect of the invention provides expression vectors comprising one or more dummy nucleic acid fragments located in the parts of the vector into which the polypeptide encoding nucleic acid inserts will be cloned, wherein said dummy fragments are positioned such that they are not in reading frame with the other parts of the expression vector and wherein said expression vectors, when expressed, do not give rise to detectable polypeptide products.

Such expression vectors are also referred to herein as dummy vectors.

Often when large expression libraries are to be generated, as in the methods of the present invention, large amounts of expression vector need to be used in the ligation reaction in which the nucleic acid fragments encoding the polypeptide fragments of interest (the inserts) are cloned into the relevant positions in the expression vectors for expression. When the vectors are prepared for ligation, usually by digesting with the appropriate restriction enzymes, there will always be some vectors which are cleaved at only one site and thus religate without gaining a new insert. This is the background of a ligation reaction and, when colonies arising from these religated vectors are screened, they may well give rise to false positive results. Where the dummy vectors of the invention are used however, the colonies arising from religated dummy vectors will not produce any detectable protein (because the dummy fragments are out of frame and the polypeptides produced will for example not contain an in frame sequence which can be detected by a detection reagent such as a tag specific antibody or another tag specific reagent) and will thus not give rise to false positives. Advantageously, this means that any polypeptides expressed and detected in the expression libraries of the invention where such vectors are used, is due to the insertion of a new and desired insert, e.g. an antibody fragment.

The dummy nucleic acid fragments are “stuffer” fragments and can be any DNA fragments providing they are positioned in the vector such that they are out of reading frame with the expression vector as a whole. Preferred dummy fragments are Green Fluorescent protein (GFP) fragments and in particular GFP fragments of approximately 600 bp or 700 bp derived from the sequence as disclosed in Genbank Accession number U55762. ID CV55762. A particularly preferred fragment of approximately 600 bp is found at positions 2-597 of the above mentioned Genbank sequence. A particularly preferred fragment of approximately 700 bp is found at positions 6-690 of the above mentioned Genbank sequence.

Preferably the dummy vectors of the invention will be used to clone antibody fragments and manufacture antibody expression libraries in accordance with the present invention as discussed above. However, it will be seen that such vectors have a wide application in the manufacture of any expression libraries where it is desired to reduce the number of false positive clones. The present invention thus further provides the use of such dummy expression vectors in the production of an expression library, e.g. an antibody expression library, and in particular the antibody expression libraries of the invention.

Preferred dummy vectors are pHOG dummy and pSEX dummy (see FIG. 2) which are designed for the production of antibody expression libraries in the scFv format. In these dummy vectors, dummy fragments of approximately 700 bp and 600 bp from GFP are inserted out of frame in the VH and VL sites, respectively, of the relevant antibody expression vectors. As shown in FIG. 2 however, dummy vectors for use in the production of antibody expression libraries in the Fab format can readily be designed and the pFAB dummy vectors as shown in FIG. 2 are also preferred.

The antibody molecules isolated, detected, selected, identified or manufactured from the expression libraries of the present invention may be used in any methods where antibodies specific to a particular antigen are required. Thus, such antibody molecules can be used as molecular tools and a further aspect of the invention provides a reagent which comprises such an antibody molecule as defined herein.

As discussed above however the antibody molecules selected or identified, etc., from the antibody expression libraries of the invention are particularly useful as therapeutic antibodies or as antibodies for in vivo diagnosis, e.g. by imaging. Other preferred uses include theranostic uses (i.e. antibodies used in both diagnosis and therapy), imaging, and as prophylactic antibodies, e.g. to confer passive immunity to particularly susceptible individuals (immunocompromised patients, small children, the fetus of pregnant women, people in endemic areas for disease, etc). Suitable and appropriate adaptations of the antibody molecules, if necessary for such uses, e.g. the conversion to IgG1 or IgG3 classes for therapy, the incorporation or addition of an appropriate label for imaging, etc., would be well known to a person skilled in the art.

The most suitable antibodies for the various uses described above can be readily identified using appropriate tests which can be designed by a person skilled in the art. For example, in applications where high affinity or avidity of an antibody is important these criteria can readily be tested in candidate antibodies using standard assay techniques (e.g. Biacore assays). In addition, where antibodies against a particular infectious agent, e.g. a bacteria, virus or fungus, have been identified, appropriate tests can be carried out to assess which antibodies are most effective to neutralize the infectious agent. For example, in the case of bacteria, bactericidal and opsonophagocytic activity against the bacteria in question can be assessed. Similarly, in the case of viruses, viral neutralisation studies can be carried out to identify the best candidates.

A yet further aspect of the invention thus provides the use of an antibody expression library as described herein to isolate, detect, identify, select or manufacture one or more antibody molecules which bind specifically to one or more target antigens, for example one or more target antigens which are associated with particular diseases or tissues or foreign agents. This could for example be carried out by screening the antibody expression libraries with the target antigens.

Yet further aspects of the invention provide such isolated, detected, identified, selected or manufactured antibody molecules for use in therapy or in vivo diagnosis or for use in any of the other applications mentioned above. Also covered is the use of such antibody molecules in the manufacture of a medicament or composition for use in therapy or in vivo diagnosis or for use in any of the other applications mentioned above. Methods of treatment of a patient comprising the administration of an appropriate dose of such an antibody molecule are also provided.

When said antibody molecules are used in the above described uses and methods then these may be administered in any appropriate way. For example such antibody molecules may be administered locally at the site where action is required or may be attached or otherwise associated with entities which will facilitate the targeting of the antibody molecules to an appropriate location in the body.

Pharmaceutical compositions comprising the antibody molecules as defined herein, together with one or more pharmaceutically acceptable carriers or excipients form a yet further aspect of the invention.

Yet further aspects are methods of diagnosis or imaging of a patient comprising the administration of an appropriate amount of an antibody molecule as defined herein to the patient and detecting the presence, location and/or amount of the antibody molecule in the patient.

The antibody molecules identified, selected, etc., from the expression libraries of the invention may equally be used in methods of diagnosis which are carried out in vitro, if appropriate, e.g. carried out on a tissue sample or some other kind of sample, e.g. blood, obtained or derived from a patient.

Preferred diseases to be treated or diagnosed, etc., are infectious diseases caused by infectious agents such as Varicella Zoster Virus, HIV, CMV, Hepatitis (and in particular Hepatitis B or C), Herpes, Meningococcus (and in particular Meningococcus A, B or C) and Streptococcus A. Particularly preferred diseases are those caused by Meningococcus B and especially preferably diseases which are stimulated by meningococcus B Outer membrane vesicles (OMV). Other preferred diseases are cancers (for example pancreatic cancers, lymphomas, sarcomas or melanomas), autoimmune diseases (for example Coeliac disease, Lupus or Rheumatoid arthritis), or allergic diseases.

The terms “therapy” or “treatment” as used herein include prophylactic therapy. The terms “therapy” and “treatment” also include combatting or cure of disease or infections or allergic reactions but also include the controlling or alleviation of disease or infection or allergic reactions or the symptoms associated therewith.

The antibodies from the expression libraries for the uses as described above are preferably IgG antibodies with a high affinity for antigen.

Yet further aspects of the present invention provide kits comprising the antibody expression libraries as defined herein or antibodies derived, selected or identified from said expression libraries. Kits comprising the dummy expression vectors of the invention are also include in the scope.

The invention will now be described in more detail in the following non-limiting Examples with reference to the following drawings in which:

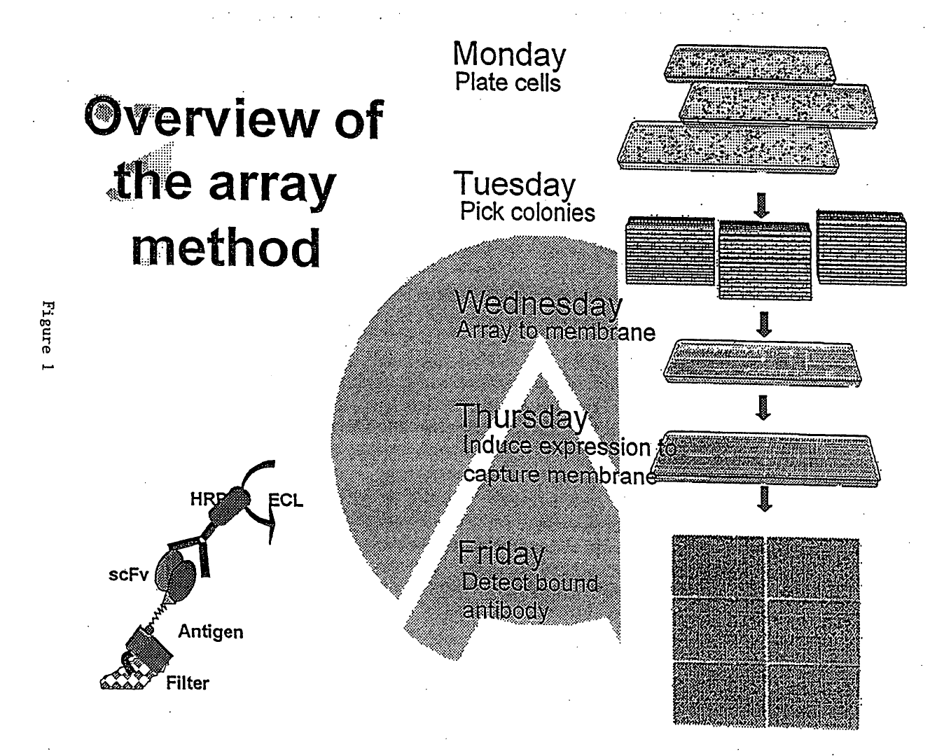

FIG. 1 shows a schematic overview of the steps of an exemplary array based screening technique which can be used to screen the expression libraries of the invention.

FIG. 2 shows schematic diagrams of the three preferred dummy expression vectors of the present invention, pHOG dummy, pSEX dummy and pFAB dummy.

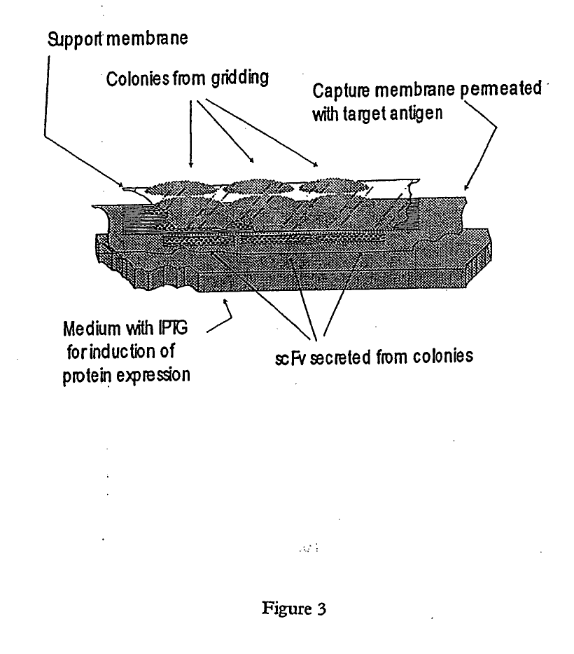

FIG. 3 shows in more detail the step of the screening technique where antibody (scFv) expression is induced and expressed proteins are captured on a capture membrane permeated with a target antigen for screening.

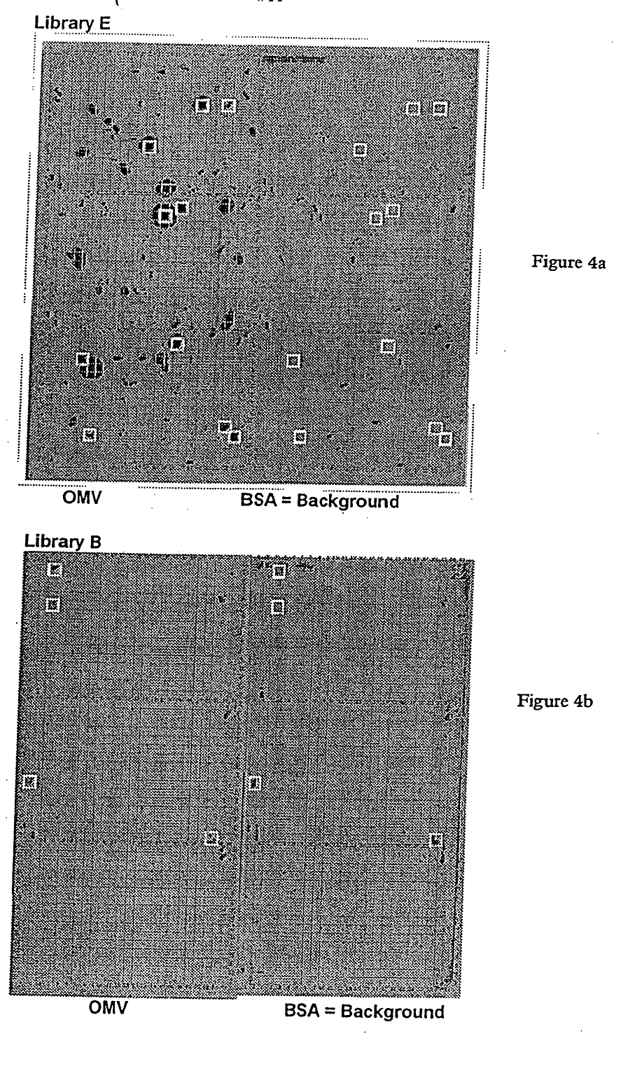

FIG. 4 shows the results of screening antibody expression libraries from four patients (patients/libraries B to E) for antibodies which interact with OMV target antigen. Positive colonies can be seen as dark spots on the OMV membranes (left hand side) compared with the BSA (background membranes). The colonies picked for further analysis are marked with white squares. FIG. 4a shows the results for patient E, FIG. 4b shows the results for patient B, FIG. 4c shows the results for patient D and FIG. 4d shows the results for patient C.

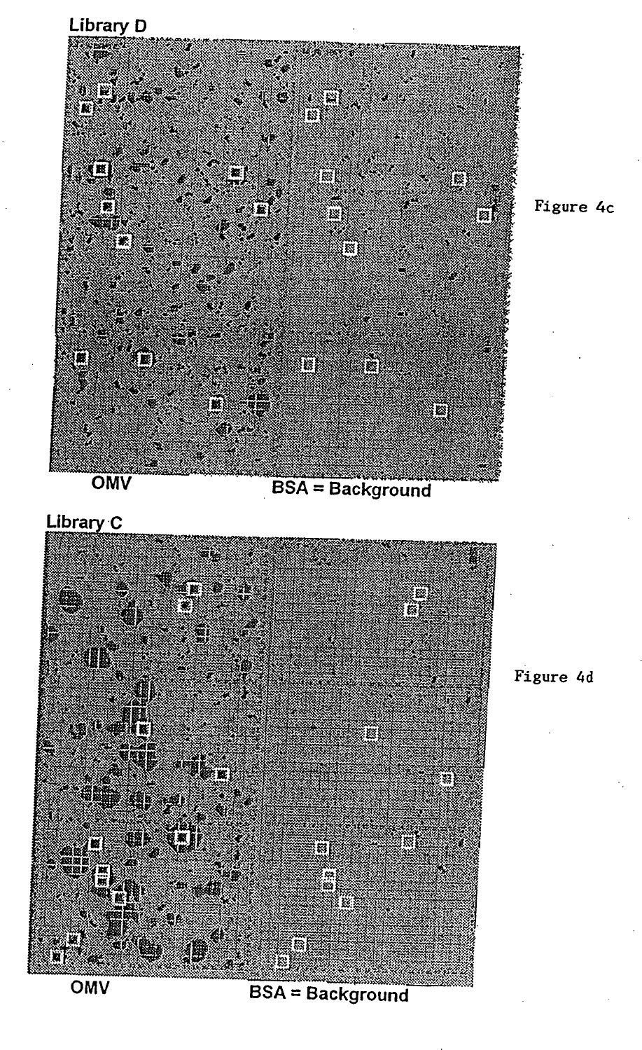

FIG. 5 shows the results of an ELISA assay to confirm that the positive and negative clones picked from patient libraries B to D were truly positive and negative for antibodies reactive with the OMV target antigen. FIG. 5a shows the results for the ten positive clones selected from patient E, FIG. 5b shows the results for the ten positive clones selected from patient D, FIG. 5c shows the results for the ten positive clones selected from patient C and FIG. 5d shows the results for the four negative clones selected from patient B.

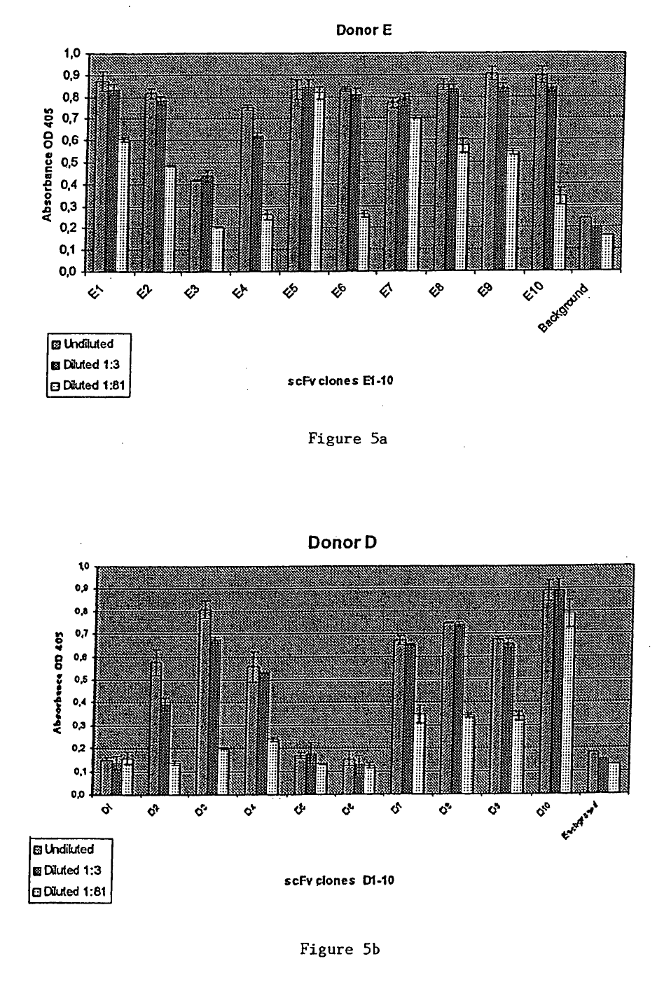

FIG. 6 shows the diversity analysis carried out on the 34 clones picked from the four patients B, C, D and E. DNA was extracted from each of the 34 clones and subjected to restriction digest with, the enzyme MvaI. The digested fragments were separated on an acrylamide gel. The results show different restriction lengths for each of the isolated plasmids indicating unique DNA sequences for each clone.

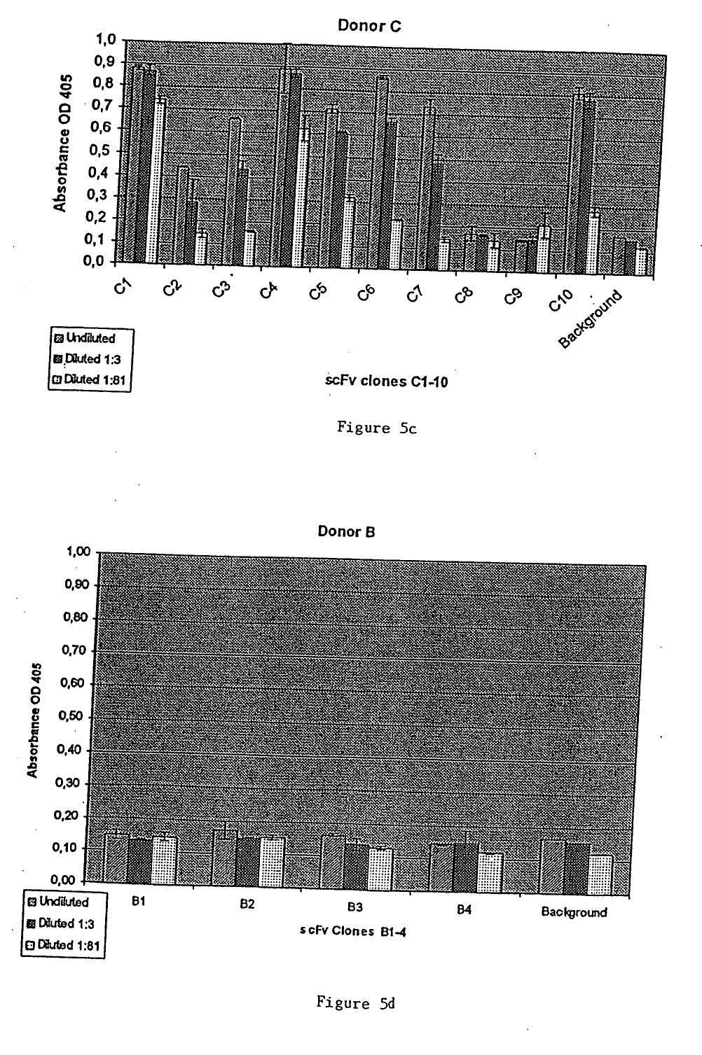

FIG. 7 shows a composite image based on films exposed to filters treated with OMV from N. meningitidis B strain 44/76 (orange), strain NZ 98/254 (purple/blue) and BSA (green). Red squares (A2, B9, C1, D3, D7, H1 and H3 from Library C, B6 and E12 from Library D and D5 from Library E) indicate signals whose associated clones were chosen for further study. Black squares (A4, B7, D2, D10, F10, F12, G10, H6 and H8 from Library C, A2, A3, A8, B2, B12, C3, C8, D3, D7 and E5 from Library D and B8, B9, C4, D1, D2, D11, D12, E1 and F5 from Library E) indicate clones that were already under investigation at the time of this screening based on their interaction with 44/76 alone.

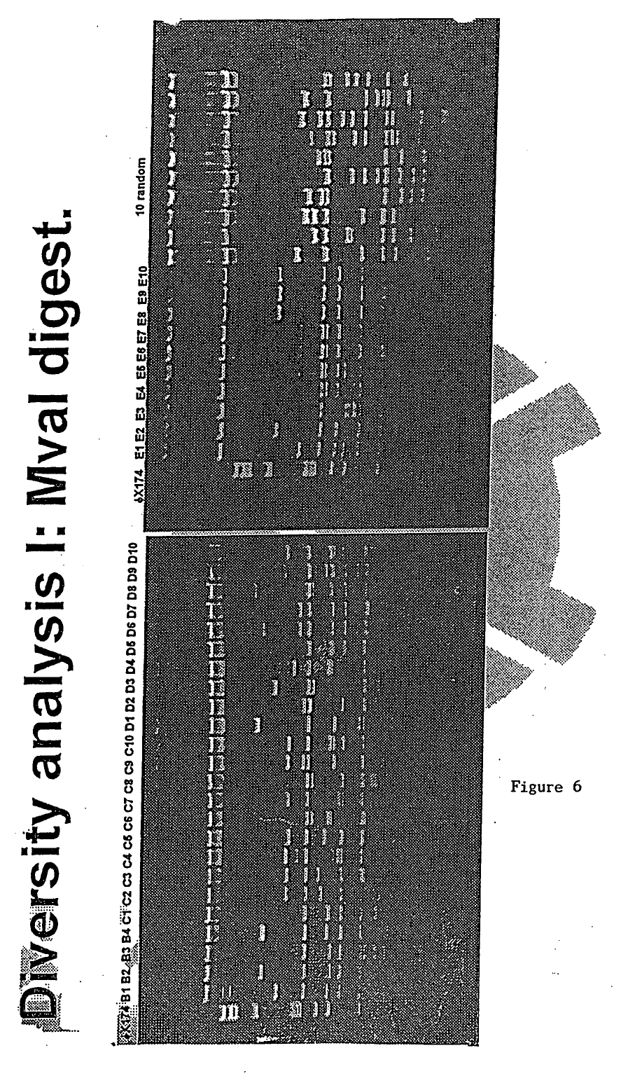

FIG. 8 shows the amino acid sequence of recombinant VZV glycoprotein E [SEQ ID NO: 3]. Boxed peptides at the amino terminal in the sequence indicate 3× FLAG tag.

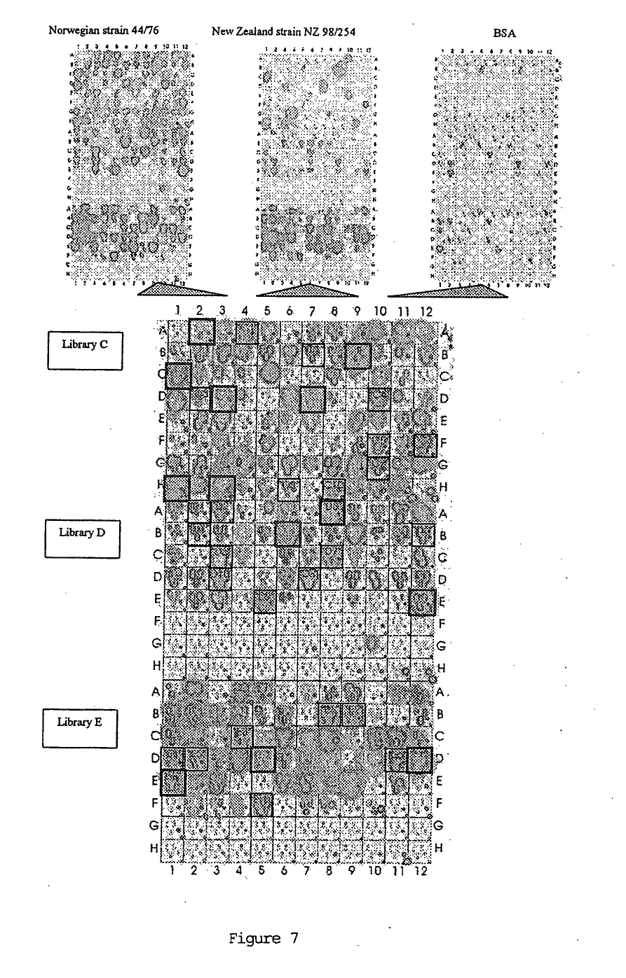

FIG. 9 shows ELISA analysis of serum from a person with no visible signs of ever having had a VZV infection (negative serum), a person who had a Varicella infection many years ago (low positive) and the serum from a patient recently infected with VZV taken 5 days (RN5) or 11 days (RN11) after detection of the first vesicular lesion.

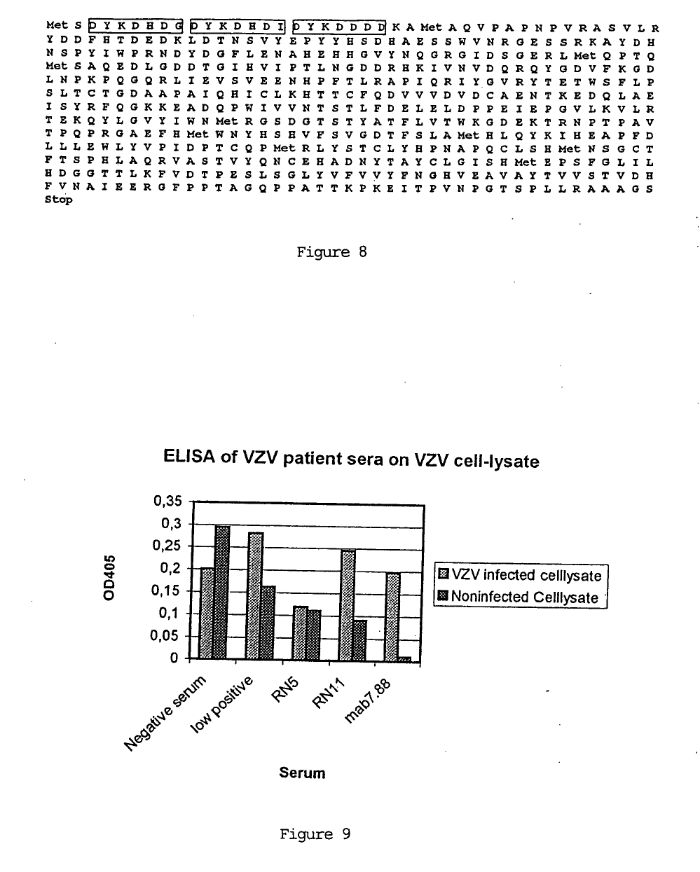

FIG. 10 shows a composite image based on films exposed to filters treated with FLAG-gE (red/purple) and BSA (green).

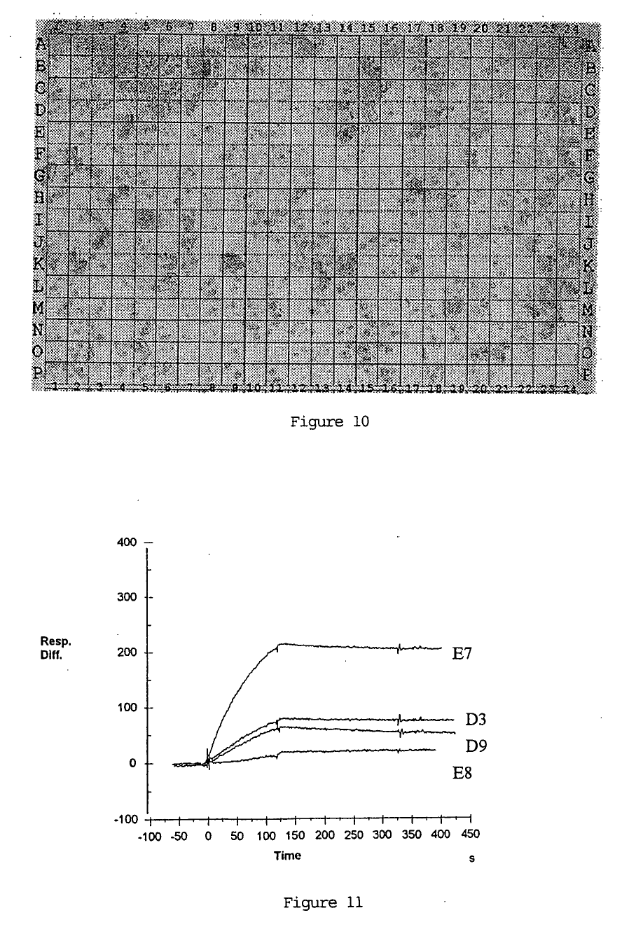

FIG. 11 shows Biacore data from selected OMV binding scFvs.

### EXAMPLE 1

Array Based Screening

Screening many thousand clones enables us to isolate antibodies directly from immunochallenged donor libraries. With the array based method (Skerra A, Dreher M L, Winter G. “Filter screening of antibody Fab fragments secreted from individual bacterial colonies: Specific detection of antigen binfind with a two-membrane system” Anal. Biochemistry 1991; 196:151-155. and de Wildt R M T, Mundy C R, Gorick B D, Tomlinson I M, “Antibody arrays for high-throughput screening of antibody-antigen interactions” Nature Biotechnology 2000; 18: 989-933) more than 10⁴ clones can be screened in one working week by one person. This method has been selected for use in the array based expression library screening carried out in the present invention, although of course other techniques could also be used.

The method is described schematically by FIG. 1. Monday: a library (of appropriate diversity) is plated on bioassay trays and incubated overnight. Tuesday: colonies grown from the plating of the library are picked to 384 well microtiter plates using a colony picking robot. Wednesday: an array is stamped from the microtiter plates to a membrane overlying nutrient agar in a bioassay tray using an arraying robot. Thursday: a second “capture” membrane is treated with the target antigen and blocked for unspecific binding. The capture membrane is then placed on agar containing IPTG. Thereafter, the first membrane with its arrays of colonies are then transferred to overlie the capture membrane. The colonies are incubated overnight and induced to express recombinant antibodies. Friday: antibodies retained on the capture membrane are detected using a secondary antibody as might be done in a Western blot. The signals produced on the filter are analysed to identify cultures containing candidate clones, which are then “cherry-picked” from the source microtiter plates.

### EXAMPLE 2

Generation and Screening of Meningococcal B Vaccine Libraries

Generation of 4 Meningococcal B Donor Libraries:

Peripheral Blood Lymphocytes (PBL) from 4 different Meningococcus B OMV (outer membrane vesicles) vaccines were isolated.

Donor B: (Aase et al., 1998, Scand. J. Immunology, vol 47:388-396). The patient was vaccinated 26/8/87 and Jun. 10, 1987 (50 μg dosages). The last dose (25 μg) was given 11/1/95. The cells were isolated 13/2/95, i.e 26 days after the last injection.

Donor C, D and E (Naess et al., 1999, Vaccine, vol 17:754-764). Patients were vaccinated with doses of 25 μg each time. Second vaccination was given 6 weeks after the first dose and the last dose was given 40 weeks after the second dose. Cells were isolated 6-7 days after the last dose.

Each patient was vaccinated with the Norwegian meningococcal B OMV vaccine 44/76 developed at the Norwegian Institute of Public Health (Fredriksen et al., supra).

RNA Isolation:

2×10⁷ cells from each donor were washed once in ice-cold PBS. The cell pellet was lysed. Between 5 to 25 μg total RNA was isolated according to the protocol from Stratagene: Strataprep® Total RNA miniprep kit.

cDNA Synthesis:

5 μg of each donor RNA was used for 1^{st} strand cDNA synthesis by the use of random hexamer oligonucleotides according to the Protocol from Gibco BRL: SuperScript Rnase H⁻ Reverse transcriptase.

| Random hexamer | 500 | ng | Promega |
|---|---|---|---|
| oligonucleotide | | | |
| Total RNA | 5 | μg | — |
| dNTP, 10 mM | 0.5 | mM | Fermentas |
| RNasin | 2 | U/μl | Promega |
| SUPERSCRIPT II RT | 200 | U | Gibco BRL |
| 5× Reaction buffer | 1× | | Gibco BRL |
| DTT, 0.1 M | 10 | mM | Gibco BRL |

RNA, dNTPs and random hexamers were incubated at 65° C. for 5 minutes followed by rapid cooling to 4° C. Reaction buffer, DTT and RNasin were added and incubated at 25° C. for 2 mins. During incubation Superscript II reverse transcriptase was added. After 10 mins the reaction was incubated at 42° C. for one hour. The Reverse transcriptase was inactivated at 70° C.
Primary Variable Gene PCR Amplification:

125 ng cDNA was used as template for the amplification of V-genes. A total of 9 IgG variable heavy chain, 20 Variable light chain kappa and 9 variable light chain lambda reactions were set up with V-gene specific oligonucleotide sets. The Reactions were run at annealing temperatures at 55, 58 or 61° C. for 30 cycles. The primer sequences used for the primary PCR-V gene amplification are shown in Table 1.

Secondary Variable Gene PCR Amplification:

2 μl of each primary PCR reaction (approximately 100 ng) was used as template for the secondary PCR reactions. A total of 45 V-Heavy, 9 V-lambda and 20 V-kappa PCR reactions were run for 30 cycles at an annealing temperature at 58° C. All the oligonucleotides introduce restriction enzyme sites, NcoI and HindIII for all V-heavy genes and MluI and NotI for all V-kappa and V-lambda genes. The primer sequences used for the secondary PCR-V gene amplification are shown in Table 2.

Digestion and Purification of Amplified Variable Genes:

All the PCR products were purified by PCR cleanup kit (Qiagen) and digested with appropriate restriction enzymes (i.e NcoI and HindIII, for V-heavy and MluI and NotI for V-kappa and lambda) overnight at 37° C. The digested fragments were run on an agarose gel and correct sized fragments were isolated and purified by Gel extraction kit (Qiagen).

Design of the pHOG, pSEX and pFAB Dummy Vectors:

pSEX and pHOG-dummy vectors were made by insertion of a 700 bp Green Fluorescent protein (GFP)-DNA fragment in the VH-site and a 600 bp GFP DNA fragment in the VL site. Both DNA fragments are cloned such that they are out of reading frame, rendering re-ligated vectors, i.e. the background in a ligation reaction, non-functional with respect to protein production. The GFP fragments of approximately 600 bp or 700 bp are derived from the sequence as disclosed in Genbank Accession number U55762. ID CV55762. The fragment of approximately 600 bp is found at positions 2-597 of the above mentioned Genbank sequence. The fragment of approximately 700 bp is found at positions 6-690 of the above mentioned Genbank sequence.

Two vector constructs were made, pHOG dummy for the expression of soluble scFv fragments and pSEX dummy for the expression of scFv fused to gpIII, a filamentous bacteriophage coat protein for the use in phagemid display of antibody fragments. Schematics showing the general structure of these dummy vectors are shown in FIG. 2. The system may also be used for the expression of Fab fragments by exchanging the VH and VL coding parts of pFAB expression vectors with GFP dummy fragments (see the schematic of the pFab dummy vector in FIG. 2).

The pHOG dummy vector is 4.3 kb and the pSEX dummy vector is 5.5 kb. These vectors also contain MYC and HIS tags located 3′ to the VL gene segment. The promoter used is the inducible LAC promoter.

Cloning of the Variable Genes into pHOG-Dummy:

The pHOG dummy vector was digested with MluI and NotI and ligated with variable light chain pools of Kappa and lambda isotypes and electroporated into XL-1 blue cells. Two light chain libraries (kappa and lambda) from each donor, total of 8 libraries, showed each between 3 to 5×10⁶ clones. The bacterial colonies were scraped from agar plates and a total plasmid pool from each library was isolated by 10 DNA miniprep (Qiagen) isolations of each.

The light chain plasmid libraries (pHOG VL) were digested with NcoI overnight at 37° C., linearised plasmid was isolated from agarose gels and purified by gel extraction kit (Qiagen) followed by secondary digestion with HinDIII overnight. The digestion reaction was incubated with calf intestinal phosphate (CIP, New England Biolabs) before the double NcoI/HinDIII digested plasmid was isolated from agarose gel and purified by gel extraction kit (Qiagen).

The NcoI and HindIII digested pHOG VL plasmids were ligated with 7 Variable heavy pools from each donor and electroporated into XL-1 blue cells. Colonies were grown on agar plates with Ampicillin (100 μl/ml) and 2 mM glucose.

Screening of 4 Meningococcal B Donor Libraries:

10 000 colonies of each library B, C, D and E were picked by the Qpix II colony picking and arraying robot (see also Example 1), i.e. a total of 40 000 colonies, and inoculated into 384-well trays with LB medium with 200 mM glucose, ampicillin (100 μg/ml), tetracycline (30 μg/ml) and 8% Glycerol. The cultures were incubated at 37° C. over night to generate master stock liquid cultures of all 40 000 clones.

The clones were gridded in an array onto a nitrocellulose membrane pre-incubated in LB/ampicillin put on top of a large LB-agar plate. The gridding was performed by the QpixII colony picker robot. The colonies were arrayed in duplicates and on three separate membranes. The colonies were allowed to grow on the nitrocellulose membrane/agar plate overnight at 30° C.

The membranes with colony arrays were put on top of a secondary nitrocellulose membrane (see FIG. 3) coated with either 450 μg OMV-target (menigococcal B Outer Membrane Vesicle) or BSA and blocked with 3% sterile filtered BSA per membrane. A third untreated membrane was included for the subsequent detection of total secreted scFv's, not shown. The colonies were induced to protein expression by IPTG (100 μM) overnight at 30° C.

The target-coated membranes (capture membranes) were washed in PBS/0.05% TWEEN (P/T). The membranes were incubated with mouse-anti-MYC antibody, Invitrogen, (1:8000 in P/T) for 1 hour at room temperature in roller-bottles. The membranes were washed in P/T for 10 mins followed by incubation with anti mouse HRP antibody, Dako, (1:10 000 in P/T) for 1 hour at room temperature in roller-bottles. The membranes were washed prior to signal development by ECL (Amersham). Positive signals were detected as spots on a light sensitive film (Amersham Hyperfilm).

Results:

From each of the donors C, D and E approximately 50 out of 10 000 clones showed positive to binding membranes coated with OMV targets and not the background membrane coated with BSA. For donor B no positives were found. From each of the donors B, C, D and E a total of 34 clones were picked, 30 positive (C1-10, D1-10 and E1-10) and 4 negative (B1-4). Picked colonies are marked with white squares on FIGS. 4a to d.

Re-Expression of Positive Anti-OMV scFv's as Soluble Antibody Fragments

Positive colonies were picked from the master stock plates and grown overnight at 37° C. in LB/Tetracyclin/Ampicillin/Glucose. Plasmids from each clone were isolated and transformed into new CaCl₂ competent XL1-blue cells. A new overnight culture from each positive clone was further inoculated into LB/TAG medium to OD 640 at 0.1. The cultures were grown to OD 640 0.6 before being induced to protein expression by changing medium to LB, ampicillin, 10 μM IPTG and incubated overnight at 30° C.

Confirmation of Positive Anti OMV scFv's in an ELISA Format.

96 well microtiter plates (NUNC, Maxisorp) were coated with 45 μg/ml OMV target overnight at 4° C. The wells were blocked with 4% skimmed milk before dilutions (undiluted, 1:3 and 1:81) of supernatant from re-expressed scFvs were added and incubated for 1 hour at room temperature. The plates were washed with P/T followed by incubation of anti-myc antibody (1:6000) for 1 hour at room temperature, after washing in P/T, anti-mouse HRP (1:10 000) was added and incubated for 1 hour at room temperature. The plates were washed in P/T and the signal was developed by adding ABTS. The signal was read at 405 nm after 20 minutes.

Of the total of 30 clones shown positive on the membrane screening, 25 were confirmed true positives in the ELISA assay (see FIGS. 5a-c). It can be seen from FIG. 5d that the four clones picked from donor B were true negatives.

DNA Analysis of Anti-OMV scFvs:

All 34 picked clones were shown by Restriction enzyme digest analysis to be unique. Plasmids derived from the 34 picked colonies were digested with restriction enzyme MvaI at 37° C. overnight. The samples were run on a 10% TBE Criterion Precast acrylamide gel (Bio-Rad). The DNA fragments are visualized by staining the gel in 0.5 mg/ml Ethidium Bromide. The results show different restriction fragment lengths among the isolated plasmids, indicating unique DNA sequences for each clone (see FIG. 6).

This means that the 25 confirmed positive anti OMV scFv's are all different and thus probably bind the same antigen at different epitopes. In other words the isolated repertoire is broad, thereby increasing the probability of isolating antibodies directed to the most effective neutralizing epitope.

Affinity Analysis of Selected Positive Anti OMV scFvs:

The affinity of the positive antibodies E7, D3, D9 and E8 as shown in FIGS. 5a and 5b for target antigen were analysed by a Biacore machine. The Biacore data for these antibodies is shown in FIG. 11. The affinities (dissociation constant, K_{D}) of the scFv fragments binding to the OMV 44/76 antigen were calculated to be:

E7=4×10⁻⁹ M

D3=6×10⁻⁹ M

D9=2×10⁻⁹ M

E8=1.5×10⁻⁸ M

These antibodies thus all show high affinity for the target antigen.

Summary

Antibody expression libraries from 4 patients vaccinated with OMV (meningococcal B outer membrane vesicles) vaccines were generated. By the process of direct screening of antibody expression libraries from three donors, many antibodies binding OMV were successfully isolated. From one donor (B) no binders were found. The main difference lies in the fact that donor B had received the vaccine for the first time 8 year prior to the third boost and that the cells were harvested 26 days after the last vaccine boost. The successful three libraries were generated from donors which had received the vaccine within one year and the cells were isolated 6-7 days after the third dose.

This indicates that the harvesting of cells from different patients especially with infectious diseases should take place within a week after outbreak or after a secondary encounter of an infectious agent. If this is done then the correct antibody producing circulating B-cells can be isolated.

Discussion:

The antibody repertoire can be screened against similar targets either to avoid or to obtain cross-reactive antibodies. For example in the course of generating human antibodies against infectious diseases the libraries can be screened against different strains of the disease causing agent. Antibodies specific for one strain must recognise a disease specific antigen. Conversely, antibodies binding different strains must recognise common antigens among the strains. At least the antibodies must recognise common or structurally similar epitopes on antigens. Such antibodies can be used as prophylactic or therapeutic antibodies against a specific strain or different strains of the disease-causing agent.

Usually vaccines are composed of attenuated virus or bacteria or composite fractions of bacteria such as capsules or membrane vesicles. Common for most vaccines are that the detailed compositions and the parts of the vaccine that are most immunogenic with respect to protective immune response are often not known, so efforts to discover good immunogens are important for the development of safe and effective vaccines. The tapping of the antibody repertoire from vaccinated individuals as described herein provides information on which immunogens foster the most effective and protective antibodies. The utilisation of a vaccinee's antibody repertoire as a tool to discover or isolate such immunogens has been proposed and termed “Reverse vaccinology” (Burton D R, Nature Reviews in Immunology 2, 706-713 (2002)). The patient antibody libraries and in particular the vaccinee antibody libraries produced in accordance with the present invention can readily be used for such reverse vaccinology for example by carrying out the following steps:
A. The vaccinee antibody repertoire is screened against the vaccine.
B. Positive antibodies binding the vaccine are isolated and used for the immunogen discovery by methods known to those skilled in the art of protein characterisation. These methods can be such as western blot, immuno-histochemistry, native gel electrophoresis, detection of protein spots on two-dimensional gel electrophoresis, isolation of protein followed by N-terminal sequencing, affinity purification of the immunogen from the vaccine. The amino acid sequence can be used to deduce the nucleic acid sequence and the native gene encoding the immunogen can be isolated by sequence homology search and PCR amplification of the gene.
C. The isolate of the gene encoding the immunogen can be produced in a suitable expression system so as to generate a pure recombinant vaccine.

An example of differential screening of OMV antibody libraries to obtain cross-reactive antibodies against two Neisseria meningitidis B strains is shown below:

Screening for Antibodies to Neisseria Meningitidis NZ-98/254 (New Zealand Strain) from Candidates Binding 44/76 (Norwegian Strain).

Selection of Candidate Clones:

Films from the initial screening against strain 44/76 (Norway) were analyzed to produce a list of candidates for screening against NZ-98/254. The films that formed the basis of the analysis were those from 10 second exposure to target filters and those from 5 minute exposure to the background filters for each of the relevant libraries (C, D and E). The criterion used in selecting candidate clones was that the clone showed clear signal on a 10 sec exposure to the target filter coated in 10 mls of 45 μg/ml (450 μg total) OMV in PBS from strain 44/76, but little or no signal on the 5 minute exposure against the background filter (coated in 10 mls 3% BSA)*. Clones that met this criterion were chosen as candidates for cross screening against NZ-98/254. From the 3 libraries, 94, 61 and 70 clones were thus chosen from libraries C, D and E respectively.

[*To facilitate the evaluation of presence versus absence of signal, images were subjected to an image analysis process. The process can be described as follows. Films were scanned and stored as “TIF” files. The files were then opened in Corel Photo Paint 9 and the “find edges” function used to define areas of signal above an arbitrary cut-off level and simultaneously define signal circumference. The images were then given color using the “replace colors” function: target images colored purple and background images green. Finally the files were exported as “GIF” files with transparent background, imported into Corel Draw 9 and superimposed and aligned such that the background (green) image was over the target (image). The superimposition of a long exposure to the background filter over a short exposure to the target filter provided a clear method for choosing candidates].

Picking of Positive Candidates, “Cherry-Picking”:

Deep well plates were filled with 400 μl HMFM (Hogness Modified Freezing Medium, 1×HMFM=3.6 mM K₂HPO₄, 1.3 mM KH₂PO₄, 2 mM Na citrate, 1 mM MgSO₄×7H₂O, 47% glycerol) per well. The Q-pix2 instrument, which employs a standard picking head, was used to “cherry-pick” from the relevant source plates to the deep well destination plates. The operation was performed in triplicate, such that each source culture was inoculated to corresponding wells in three separate destination plates. Cultures were grown 20 hours at 37° C. with shaking. Once 384 well plates were prepared from the 96 well deep well plates (see next section) the cultures were frozen at −80° C.

Preparing 384 Well Plates for Arraying of Candidate Clones:

To array the cherry-picked clones with our equipment it was necessary to transfer an aliquot of each culture in the 96 deep-well plates to a well in a 384 well plate. Therefore, a 384 well plate was prepared for each of the 3 replicate picks mentioned above (resulting in 9 plates: library C rep1, rep2 rep3, library D rep1 rep2 rep3, library E rep1, rep2 rep3).

Arraying:

The arraying strategy was chosen in a way to “cluster” replicate clones. This was achieved by choosing the appropriate plate placement and 3×3 pattern. The pattern chosen included duplicate printing of each spot within its subgrid. Each clone was thus represented as duplicate printings of triplicate cherry-pickings (see Table 3). Arrays were printed from the 384 well plates described above on to blocked filters placed on growth medium in 22×22 cm bioassay trays. The filters employed were 22×22 cm Schleiser and Schuell “Protan” cellulose nitrate filters, pore size 0.45 μm, and these were blocked in 50 ml 2% BSA (1 g total) for 1 hr. The growth medium employed was 200 ml LB agar containing 100 μg/ml ampicillin 30 μg/ml tetracycline. Colonies were allowed to grow at 37° C. for 16 hrs.

TABLE 3
Correspondence between well A1 in the deep-well plates inoculated
from the cherry-pick, the 384 well plates used to create the
array and the position of colonies on the array*

| | |
|---|---|
| 96 deep-well plates | 1 |

384 well plates used in arraying

| | | pattern of colonies | | | | | | |
|---|---|---|---|---|---|---|---|---|
| | | in array | subgr. col. 1 | | | subgr. col. 2 | | |
| | | | c | | 3 | c | | 3 |
| | A | subgrid row A | 2 | 1 | 3 | 2 | 1 | 3 |
| | | | 2 | 1 | c | 2 | 1 | c |
| A | | | | | | | | |
| | | | c | | 3 | c | | 3 |
| | B | subgrid row B | 2 | 1 | 3 | 2 | 1 | 3 |
| | | | 2 | 1 | c | 2 | 1 | c |

[*The cells at the bottom right of the table show the 3 by 3 pattern of duplicates used in the arraying procedure. Those positions marked in gray font are those printed from empty wells, whereas those in bold indicate wells containing cultures from the cherry-picking procedure. Subgrid row A, column 1 was thereby occupied only by two colonies, each from replicate 1. Similarly, subgrids A2, and B1 were occupied only by duplicate colonies of replicates 2 and 3 respectively. Also, the constitutive positive control clone (“c”) was printed to subgrid B2.]
Capture Filter Coating:

Two “target” filters were prepared. The primary target filter was prepared from the New Zealand strain NZ-98/254 of N. meningitidis OMV (outer membrane vesicles). The secondary filter was that prepared from OMV of the Norwegian strain 44/76. One “background” filter was also prepared by coating only with block agent (3% BSA). The coating procedure was performed as follows. Schleiser and Schuell “Protan” cellulose nitrate filters, pore size 0.45 μm, were cut to three 11 by 22 cm strips and treated as follow. Filters were first wet in PBS, rolled into nylon mesh and inserted into large (1.5 liter) hybridization tubes. The target filters were then incubated with 25 ml of 45 μg/ml OMV (1.125 mg total) in PBS for 1 hr, while the background filter was incubated with PBS. The filters were then drained and incubated with 25 ml 3% BSA (750 mg total) in PBS for 1 hr to block. Filters were then drained and rinsed in 50 ml LB before application to the induction plate.

Induction-Capture and Detection Conditions:

Induction-capture and detection was performed for each of four capture filters (2 “target” filters coated either with NZ 98/254 or 44/76, 1 background filter coated with BSA and 1 untreated filter used to monitor scFv expression). Induction-capture were achieved by superimposing the filter bearing the colonies grown in the arraying process onto the capture filter on an induction plate. Induction plates were comprised of 200 mls LB agar containing 100 μg/ml ampicillin, 30 μg/ml tetracycline and 100 mM IPTG in 22×22 cm bioassay trays. The induction-capture “sandwich” was incubated 18 hrs at 30° C., allowing secreted antibody fragments (scFv) to be expressed and secreted from the colonies and to diffuse to the capture filter. Colony bearing filters were then removed and capture filters subjected to the detection procedure. The detection procedure can be described as follows: wash 3×5 min in 50 ml PBS 0.05% Tween; incubate 1 hr with 25 ml mouse anti c-myc (1:10 000); wash 3×5 min in 50 ml PBS 0.05% Tween; incubate 1 hr with 25 ml goat anti-mouse HRP (1:10 000); wash 5×5 min in 50 ml PBS 0.05% Tween; use ECL (Amersham) reagents to produce autoradiograms to film. For each filter, exposures to film were made at 10, 45 and 120 second durations.

Evaluation of Images to Identify Cross-Binding Clones:

As in the initial screening of clones, images were analysed with the help of functions in Corel Photo Paint 9. Specifically, films were scanned and files imported into Corel Photo Paint 9 where the “edge detect” function was used to define a radius of density around the spots produced on film during the detection procedure. The function uses local differences in density to establish the presence of “edges”, thereby providing an objective criterion for spot presence: spots under a certain threshold level are lost from the image. After detection of edges, images were colored according to their source film: orange=10 sec exposure 44/76 coated filter; purple=10 sec exposure to NZ 98/254 coated filter; blue=45 sec exposure to NZ 98/254 coated filter; green=120 sec exposure to filter blocked with BSA only. Images were exported as “transparent” gifs and layered orange, blue, purple, green from bottom to top (see FIG. 7). Candidate clones were chosen based on corresponding purple/blue spots (indicating binding to NZ 98/254) that were clearly larger than the spots originating from the BSA only (background) filter. Also, one candidate was chosen that showed strong binding to 44/76, but no detectable interaction with NZ 98/254 (Clone D5 from library E—see FIG. 7). The 10 clones chosen in this screening then are those listed in Table 4 below and indicated by red boxes in FIG. 7. Note that prior to the screening against strain NZ 98/254, 30 additional clones were chosen based on their affinity for strain 44/76 alone (see FIGS. 5a, b and c). About 10% of the tested clones are cross reactive.

The 10 clones chosen in this screening cross react with the OMV from both the NZ 98/254 and Norwegian 44/76 strains of N. meningitidis B. The antibodies of these clones are good candidates for broad range high affinity propylactic or therapeutic antibodies against N. meningitidis B and in particular the strains NZ 98/254 and Norwegian 44/76. In addition, these antibodies can be used in reverse vaccinology as described above in order to identify the particular immunogen in the vaccines which are recognised by these antibodies and the gene encoding this immunogen.

Diversity of the Repertoire

The diversity of the repertoire of antibody molecules which can be derived from the patient libraries of the invention was demonstrated by selecting 33 positive clones from libraries C, D and E and analysing the variable regions of the ScFv fragments from these clones. These results are shown in Table 5 where it can be seen that the variable heavy chain (VH) fell into the VH1, 3 and 4 families. The variable light chains (VL) were comprised by Vκ (kappa) 1 and 3, Vλ (lambda) 1, 2, 3, 6 and 8 families thus representing a typical level of V-gene usage among humans.

In carrying out this analysis all variable genes were sequenced with oligonucleotides annealing to the 5′ and the 3′ ends of the Variable Heavy and variable light chain genes respectively. Sequencing was performed at GATC biotech AG, Constanz, Germany. To identify the V-gene family all v-gene sequences were analysed by DNAPLOT at the V-base (http://www.mrc-cpe.cam.ac.uk).

### EXAMPLE 3

Generation and Screening of a Varicella Zoster Virus Patient Antibody Library

Discussion:

Whereas Example 2 is based on antibody libraries generated from individuals vaccinated against a bacterial disease, the following Example is based on an antibody library from a primary infected patient with a viral disease, Varicella zoster virus showing that the process also is valid for viral diseases and that antibody libraries from real patients can successfully be screened.

Generation of Recombinant VZV GlycoproteinE:

DNA sequence information of gE was obtained from EMBL: HEVZVXX, accession X04370 (ref: Davidson A J and Scott J E The complete DNA sequence of varicella-zoster virus. J Gen Virol 9, 1759-1816, 1986). Oligonucleotides for the amplification of glycoprotein E extracellular part were designed:

1: 5′ AGAGAGCAGCTGCGTATAACGAATCCGGTCAGA [SEQ ID NO: 1]

2: 5′ AGAGAGGCGGCCGCTCGTAGAAGTGGTGACGT. [SEQ ID NO: 2]

The PCR product includes restriction enzyme cleavage sites for PvuI and NotI in oligonucleotide 1 and 2 respectively (underlined). The PCR product was digested with PvuI and NotI enzymes and cloned into a version of pHOG dummy in which the MYC and HIS tag is removed but with a FLAG tag inserted N-terminally to the gE protein. The clone was sequenced and verified correct native gE within the desired sequence (see FIG. 8 gE protein sequence). Recombinant FLAG-gE was expressed in E. coli. By the denaturation and renaturation of gE inclusion bodies, soluble, recombinant gE was produced. The recombinant gE was verified structurally functional by positive binding of gE specific monoclonal antibodies (data not shown).

Generation of VZV Cell Lysate and Control Antigen:

Low passage virus was used for infection of HE cells (human embryonic fibroblasts). Cells should not have more than 17 passages. 0.5 to 1.5 ml virus incubated/absorbs the cells at 1-1.5 hours at room temperature (RT). Eagle's minimal essential medium (EMEM) supplemented with penicillin and Streptomycin, 10% FBS and 1 ml 5% NaHCO₃/100 ml medium was added and further incubated at 37° C. overnight (ON). Medium was changed after 24 hours. The cells were harvested after 52 hours. The cells were sonicated for 15 seconds and centrifuged at 1000 rpm. Non-infected cells were treated in an identical way for the generation of control antigen.

Generation of VZV Patient Antibody Library:

PBL was taken at day 5 and day 11 after appearance of the first vesicular lesion on an adult male with primary varicella infection.

10 ml of PBL was spun 4000 rpm for 15 minutes and the serum was frozen in −20° C.

Isolation of Lymphocytes:

The lymphocytes were isolated using the Lymphoprep™ kit (Axis-Shield). 4×10 ml batches of the PBL was mixed with 10 ml PBS and gently layed over 10 ml of lymphoprep. The mixture was centrifuged for 50 minutes at 800 g, room temperature, before the sample/medium interface of mononuclear cells was removed, using a pasteur pipette. The mononuclear cells were washed two times in PBS. The cells were counted and frozen in batches of 5×10⁷ cells.

ELISA Control of Anti-VZV Serum:

Microtiterplates were coated with 4 μg/ml of both VZV cell lysate and control antigen. All sera were diluted 1:50 in water and 100 μl of each diluted sera was added to both the VZV cell lysate and control antigen coated microtiter wells.

The negative serum was from a person with no visible signs of ever having had a VZV infection. The positive control is from a person who had a Varicella infection many years ago (low positive), and still displays a certain level of antibodies in the PBL. Mab7.88 is a mouse monoclonal antibody (National Institute of Public Health, Norway) diluted 1:1000 before use. The ELISA results (FIG. 9) show that there are no detectable anti VZV IgG antibodies in the sera of the patient from day 5 (RN5), but at day 11 (RN11) we can show an IgG titer against the VZV infected cell-lysate. Based on these results the library was made from the lymphocytes isolated on day 11 after detection of the first vesicular lesion.

The VZV antibody library was generated as described in Example 2.

Plating and Picking:

Transformation mix was plated on LB agar containing 100 μg/ml ampicillin and 30 μg/ml tetracycline in 22×22 cm bioassay trays (200 ml) and incubated at 37° C. for 16 hrs. Colonies were then picked to 384 well plates containing 80 μl per well of LB with 8% glycerol, 100 μg/ml ampicillin and 30 μg/ml tetracycline. Colonies to fill eight 384 well plates were picked (potentially 3072 cultures). Cultures were incubated 18 hrs at 37° C.

Arraying:

Arrays were printed using the Q-pix 2 in a 4×4 pattern from the 384 well plates produced in the picking procedure described in Example 2. These were printed on to blocked filters placed on growth medium in 22×22 cm bioassay trays. The filters employed were 22×22 cm Schleiser and Schuell “Protan” cellulose nitrate filters, pore size 0.45 μm, and these were blocked in 50 ml 2% BSA (1 g total) for 1 hr. The growth medium employed was 200 ml LB agar containing 100 μg/ml ampicillin 30 μg/ml tetracycline. Colonies were allowed to grow at 37° C. for 16 hrs.

Capture Filter Coating:

The target filter was coated with the recombinant FLAG gE-protein. A “background” filter was also prepared by coating only with block agent (3% BSA). The coating procedure was performed as follows. Schleiser and Schuell “Protan” cellulose nitrate filters, pore size 0.45 μm, were cut to three 11 by 7 cm sections and treated as follows. Filters were first wet in PBS, rolled into nylon mesh and inserted into large (1.5 liter) hybridization tubes. The target filter was then incubated with 8 ml of 375 μg/ml FLAG-gE-protein (3 mg total) in PBS for 1 hr, while the background filter was incubated with PBS. The filters were then drained and incubated with 8 ml 3% BSA (240 mg total) in PBS for 1 hr to block. Filters were then drained and rinsed in 50 ml LB before application to the induction plate.

Induction-Capture and Detection Conditions:

Induction-capture and detection was performed for each of three capture filters (1 “target” filter coated with FLAG-gE, 1 background filter coated with BSA and 1 untreated filter used to monitor scFv expression). Induction-capture was achieved by superimposing the filter bearing the colonies grown in the arraying process onto the capture filter on an induction plate. Induction plates were comprised of 200 mls LB agar containing 100 μg/ml ampicillin, 30 μg/ml tetracycline and 100 mM IPTG in 22×22 cm bioassay trays. The induction-capture “sandwich” was incubated 17.5 hrs at 30° C., allowing secreted antibody fragments (scFv) to be expressed and secreted from the colonies and to diffuse to the capture filter. Colony bearing filters were then removed and capture filters subjected to the detection procedure. The detection procedure can be described as follows: wash 3×5 min in 50 ml PBS 0.05% Tween; incubate 1 hr with 25 ml mouse anti c-myc (1:10 000); wash 3×5 min in 50 ml PBS 0.05% Tween; incubate 1 hr with 25 ml goat anti mouse HRP (1:10 000); wash 5×5 min in 50 ml PBS 0.05% Tween; use ECL (Amersham) reagents to produce autoradiograms to film. For each filter, exposures to film were made at 1 and 2 minute durations.

Evaluation of Images to Identify Clones Binding with Specific Affinity for gE:

Images were analysed with the help of functions in Corel Photo Paint 9 in a way similar to that described in Example 2.

TABLE 1
Primer Sequences for primary PCR V-gene amplification¹

| | | | V-fam. | Tm |
|---|---|---|---|---|
| No | Name | Sequence (5′ - 3′) | recog. | (NN/%/R) |
| N1 | VH4back1 | CAG GTG CAG CTG CAG GAG TCC G | 4 | 71.89/76.9/74.00 |
| | | [SEQ ID NO: 4] | | |
| N2 | VH4back2 | CAG GTG CAG CTG CAG GAG TCG G | 4 | 71.89/76.9/74.00 |
| | | [SEQ ID NO: 5] | | |
| N3 | VH5back | CAG GTA CAG CTG CAG CAG TCA | 6 | 62.74/72.8/66.00 |
| | | [SEQ ID NO: 6] | | |
| N4 | VH6back | CAG GTG CAG CTA CAG CAG TGG G | 4 | 67.57/76.9/72.00 |
| | | [SEQ ID NO: 7] | (DP63) | |
| N5 | VH10back | GAG GTG CAG CTG KTG GAG WCY | 3 | 66.81/72.8/70.00 |
| | | [SEQ ID NO: 8] | | |
| N6 | VH12back | CAG GTC CAG CTK GTR CAG TCT GG | 1 | 70.73/75.3/76.00 |
| | | [SEQ ID NO: 9] | | |
| N7 | VH14back1 | CAG ATC ACC TTG AAG GAG TCT G | 2 | 61.23/73.2/66.00 |
| | | [SEQ ID NO: 10] | | |
| N8 | VH14back2 | CAG GTC ACC TTG AAG GAG TCT G | 2 | 61.23/71.3/68.00 |
| | | [SEQ ID NO: 11] | | |
| N9 | VH22back | CAG GTG CAG CTG GTG SAR TCT GG | 1, 2, | 71.77/75.3/76.00 |
| | | [SEQ ID NO: 12] | 5, 7 | |
| N10 | VL1back | CAG TCT GTS BTG ACG CAG CCG CC | 1 | 75.34/77.1/78.00 |
| | | [SEQ ID NO: 13] | | |
| N11 | VL3back | TCC TAT GWG CTG ACW CAG CCA C | 3 | 62.71/73.2/68.00 |
| | | [SEQ ID NO: 14] | | |
| N12 | VL38back | TCC TAT GAG CTG AYR CAG CYA CC | 3 | 69.93/71.8/74.00 |
| | | [SEQ ID NO: 15] | | |
| N13 | VL4back | CAG CCT GTG CTG ACT CAR YC | 1, 4, | 64.43/70.3/66.00 |
| | | [SEQ ID NO: 16] | 5, 9 | |
| N14 | VL7/8back | CAG DCT GTG GTG ACY CAG GAG CC | 7, 8 | 73.35/77.1/78.00 |
| | | [SEQ ID NO: 17] | | |
| N15 | VL9back | CAG CCW GKG CTG ACT CAG CCM CC | 1, 5, | 73.55/78.9/80.00 |
| | | [SEQ ID NO: 18] | 9, 10 | |
| N16 | VL11back | TCC TCT GAG CTG AST CAG GAS CC | 3 | 66.57/73.5/74.00 |
| | | [SEQ ID NO: 19] | (DPL16) | |
| N17 | VL13back | CAG TCT GYY CTG AYT CAG CCT | 2 | 64.33/68.9/68.00 |
| | | [SEQ ID NO: 20] | | |
| N18 | VL15back | AAT TTT ATG CTG ACT CAG CCC C | 6 | 61.65/69.5/64.00 |
| | | [SEQ ID NO: 21] | | |
| N19 | VK1back | GAC ATC CRG DTG ACC CAG TCT CC | 1 | 70.32/75.3/76.00 |
| | | [SEQ ID NO: 22] | | |
| N20 | VK2back | GAA ATT GTR WTG ACR CAG TCT CC | 3, 6 | 63.16/68.2/68.00 |
| | | [SEQ ID NO: 23] | | |
| N21 | VK9back | GAT ATT GTG MTG ACB CAG WCT CC | 2, 3, | 62.13/70.0/70.00 |
| | | [SEQ ID NO: 24] | 4, 6 | |
| N22 | VK12back | GAA ACG ACA CTC ACG CAG TCT C | 5 | 62.36/73.2/68.00 |
| | | [SEQ ID NO: 25] | | |
| N23 | VH1/2for1 | TGA GGA GAC AGT GAC CAG GGT G | JH1, | 64.94/75.0/70.00 |
| | | [SEQ ID NO: 26] | JH2 | |
| N24 | VH1/2for2 | TGA GGA GAC GGT GAC CAG GGT G | JH1, | 69.11/76.9/72.00 |
| | | [SEQ ID NO: 27] | JH2 | |
| N25 | VH4/5for | TGA GGA GAC GGT GAC CAG GGT T | JH4, | 67.49/75.0/70.00 |
| | | [SEQ ID NO: 28] | JH5 | |
| N26 | VH3for | TGA AGA GAC GGT GAC CAT TGT | JH3 | 60.56/68.9/32.00 |
| | | [SEQ ID NO: 29] | | |
| N27 | VH6for | TGA GGA GAC GGT GAC CGT GGT CC | JH6 | 71.96/78.9/76.00 |
| | | [SEQ ID NO: 30] | | |
| N28 | IgMFor | GGT TGG GGC GGA TGC ACT CC | CH1 | 71.31/76.5/68.00 |
| | | [SEQ ID NO: 31] | Cμ | |
| N29 | IgG For | SGA TGG GCC CTT GGT GGA RGC | CH1 | 73.00/74.7/72.00 |
| | | [SEQ ID NO: 32] | Cγ | |
| N30 | VL1/2for | TAG GAC GGT SAS CTT GGT CC | Jλ1, | 62.26/68.3/64.00 |
| | | [SEQ ID NO: 33] | Jλ2, | |
| | | | Jλ3 | |
| N31 | VL7for | GAG GAC GGT CAG CTG GGT GC | Jλ7 | 67.82/76.5/68.00 |
| | | [SEQ ID NO: 34] | | |
| N32 | VK1for | TTT GAT TTC CAC CTT GGT CC | Jκ1 | 59.77/66.2/58.00 |
| | | [SEQ ID NO: 35] | | |
| N33 | VK2/4for1 | TTT GAT CTC CAC CTT GGT CC | Jκ2, | 59.90/68.3/60.00 |
| | | [SEQ ID NO: 36] | Jκ4 | |
| N34 | VK2/4for2 | TTT GAT CTC CAG CTT GGT CC | Jκ2, | 60.20/68.3/60.00 |
| | | [SEQ ID NO: 37] | Jκ4 | |
| N35 | VK3for | TTT GAT ATC CAC TTT GGT CC | Jκ3 | 54.97/64.2/56.00 |
| | | [SEQ ID NO: 38] | | |
| N36 | VK5for | TTT AAT CTC CAG TCG TGT CC | Jκ5 | 55.20/66.2/58.00 |
| | | [SEQ ID NO: 39] | | |
| N37* | IgAfor1 | AGA CCT TGG GGC TGG TCG GGG | CH1 | 72.63/78.6/72.00 |
| | | [SEQ ID NO: 40] | Cα | |
| N38* | IgAfor2 | GAG GCT CAG CGG GAA GAC CTT | CH1 | 66.37/74.7/68.00 |
| | | [SEQ ID NO: 41] | Cα | |
| N39* | IgEfor1 | GAG GTG GCA TTG GAG GGA ATG | CH1 | 66.04/72.8/66.00 |
| | | [SEQ ID NO: 42] | Cε | |
| N40^{□} | IgEfor2 | GAC GGA ATG GGC TCG TGT GGA | CH1 | 69.87/74.7/68.00 |
| | | [SEQ ID NO: 43] | Cε | |
| N41^{□} | IgDfor | CAC ATC CGG AGC CTT GGT GGG | CH1 | 71.18/76.7/70.00 |
| | | [SEQ ID NO: 44] | Cδ | |

B = C, G, T
D = A, D = A, G, T
R = A, G
S = C, G
NN = nearest neighbour
(www.williamstone.com)
K = G, T
M = A, C
W = A, T
Y = C, T
% = % GC (Oligo Tech)
(www.williamstone.com)
R = 2(A + T) + 4(G + C)
¹Sblattero, D. & Bradbury, A. A definitive set of oligonucleotide primers for amplifying human V regions. Immunotechnology 3, 271-8 (1998).
*Ole H. Brekke
^{□}Petra (Heidelberg)

TABLE 2

Primer sequences for secondary PCR V-gene
amplification

| | | | |
|---|---|---|---|
| No | Name | Sequence (5′ - 3′) | RE tag |

| | | | |
|---|---|---|---|
| N1T | VH4back1N | AG AGA GCC ATG GCC CAG GTG | Nco I |
| | | CAG CTG CAG GAG TCC G | |
| | | [SEQ ID NO: 45] | |
| N2T | VH4back2N | AG AGA GCC ATC GCC CAG GTG | Nco I |
| | | CAG CTG CAG GAG TCG G | |
| | | [SEQ ID NO: 46] | |
| N3T | VH5backN | AG AGA GCC ATG GCC CAG GTA | Nco I |
| | | CAG CTG CAG CAG TCA | |
| | | [SEQ ID NO: 47] | |
| N4T | VH6backN | AG AGA GCC ATG GCC CAG GTG | Nco I |
| | | CAG CTA CAG CAG TGG G | |
| | | [SEQ ID NO: 48] | |
| N5T | VH10backN | AG AGA GCC ATG GCC GAG GTG | Nco I |
| | | CAG CTG KTG GAG WCY | |
| | | [SEQ ID NO: 49] | |
| N6T | VH12backN | AG AGA GCC ATG GCC CAG GTC | Nco I |
| | | CAG CTK GTR CAG TCT GG | |
| | | [SEQ ID NO: 50] | |
| N7T | VH14back1N | AG AGA GCC ATG GCC CAG ATC | Nco I |
| | | ACC TTG AAG GAG TCT G | |
| | | [SEQ ID NO: 51] | |
| N8T | VH14back2N | AG AGA GCC ATG GCCCAG GTC | Nco I |
| | | ACC TTG AAG GAG TCT G | |
| | | [SEQ ID NO: 52] | |
| N9T | VH22backN | AG AGA GCC ATG GCC CAG GTG | Nco I |
| | | CAG CTG GTG SAR TCT GG | |
| | | [SEQ ID NO: 53] | |
| N10T | VL1backM | A GAG AGA CGC GTA CAG TCT | Mlu I |
| | | GTS BTG ACG CAG CCG CC | |
| | | [SEQ ID NO: 54] | |
| N11T | VL3backM | A GAG AGA CGC GTA TCC TAT | Mlu I |
| | | GWG CTG ACW CAG CCA C | |
| | | [SEQ ID NO: 55] | |
| N12T | VL38backM | A GAG AGA CGC GTA TCC TAT | Mlu I |
| | | GAG CTG AYR CAG CYA CC | |
| | | [SEQ ID NO: 56] | |
| N13T | VL4backM | A GAG AGA CGC GTA CAG CCT | Mlu I |
| | | GTG CTG ACT CAR YC | |
| | | [SEQ ID NO: 57] | |
| N14T | VL7/8backM | A GAG AGA CGC GTA CAG DCT | Mlu I |
| | | GTG GTG ACY CAG GAG CC | |
| | | [SEQ ID NO: 58] | |
| N15T | VL9backM | A GAG AGA CGC GTA CAG CCW | Mlu I |
| | | GKG CTG ACT CAG CCM CC | |
| | | [SEQ ID NO: 59] | |
| N16T | VL11backM | A GAG AGA CGC GTA TCC TCT | Mlu I |
| | | GAG CTG AST CAG GAS CC | |
| | | [SEQ ID NO: 60] | |
| N17T | VL13backM | A GAG AGA CGC GTA CAG TCT | Mlu I |
| | | GYY CTG AYT CAG CCT | |
| | | [SEQ ID NO: 61] | |
| N18T | VL15backM | A GAG AGA CGC GTA AAT TTT | Mlu I |
| | | ATG CTG ACT CAG CCC C | |
| | | [SEQ ID NO: 62] | |
| N19T | VK1backM | A GAG AGA CGC GTA GAC ATC | Mlu I |
| | | CRG DTG ACC CAG TCT CC | |
| | | [SEQ ID NO: 63] | |
| N20T | VK2backtsM | A GAG AGA CGC GTA GAA ATT | Mlu I |
| | | GTR WTG ACR CAG TCT CC | |
| | | [SEQ ID NO: 64] | |
| N21T | VK9backM | A GAG AGA CGC GTA GAT ATT | Mlu I |
| | | GTG MTG ACB CAG WCT CC | |
| | | [SEQ ID NO: 65] | |
| N22T | VK12backM | A GAG AGA CGC GTA GAA ACG | Mlu I |
| | | ACA CTC ACG CAG TCT C | |
| | | [SEQ ID NO: 66] | |
| N23T | VH1/2for1H | AGA GAG AAG CTT TGA GGA | Hind III |
| | | GAC AGT GAC CAG GGT G | |
| | | [SEQ ID NO: 67] | |
| N24T | VH1/2for2H | AGA GAG AAG CTT TGA GGA | Hind III |
| | | GAC GGT GAC CAG GGT G | |
| | | [SEQ ID NO: 68] | |
| N25T | VH4/5forH | AGA GAG AAG CTT TGA GGA | Hind III |
| | | GAC GGT GAC CAG GGT T | |
| | | [SEQ ID NO: 69] | |
| N26T | VH3forH | AGA GAG AAG CTT TGA AGA | Hind III |
| | | GAC GGT GAC CAT TGT | |
| | | [SEQ ID NO: 70] | |
| N27T | VH6forH | AGA GAG AAG CTT TGA GGA | Hind III |
| | | GAC GGT GAC CGT GGT CC | |
| | | [SEQ ID NO: 71] | |
| N30T | VL1/2forN | AG AGA GGC GGC CGC TAG | Not I |
| | | GAC GGT SAS CTT GGT CC | |
| | | [SEQ ID NO: 72] | |
| N31T | VL7forN | AG AGA GGC GGC CGCGAG | Not I |
| | | GAC GGT CAG CTG GGT GC | |
| | | [SEQ ID NO: 73] | |
| N32T | VK1/2forN | AG AGA GGC GGC CGC TTT | Not I |
| | | GAT TTC CAC CTT GGT CC | |
| | | [SEQ ID NO: 74] | |
| N33T | VK2/4for1N | AG AGA GGC GGC CGC TTT | Not I |
| | | GAT CTC CAC CTT GGT CC | |
| | | [SEQ ID NO: 75] | |
| N34T | VK2/4for2N | AG AGA GGC GGC CGC TTT | Not I |
| | | GAT CTC CAG CTT GGT CC | |
| | | [SEQ ID NO: 76] | |
| N35T | VK3forN | AG AGA GGC GGC CGC TTT | Not I |
| | | GAT ATC CAC TTT GGT CC | |
| | | [SEQ ID NO: 77] | |
| N36T | VK5forN | AG AGA GGC GGC CGC TTT | Not I |
| | | AAT CTC CAG TCG TGT CC | |
| | | [SEQ ID NO: 78] | |

B = C, G, T
D = A, G, T
K = G, T
M = A, C
R = A, G
S = C, G
W = A, T
Y = C, T

TABLE 5
The V-gene families and specificity of 33 positive
ScFv fragments from libraries C, D and E.

| Clone ID | VH Family | VL Family | Specificity |
|---|---|---|---|
| C1 | VH3 | nd | 44/76 |
| C2 | nd | Vκ2 | 44/76 |
| C3 | VH3 | Vλ3 | 44/76 |
| C4 | VH3 | Vλ3 | 44/76 |
| C5 | nd | Vλ3 | 44/76 |
| C6 | VH3 | Vλ2 | 44/76 |
| C10 | VH3 | Vκ1 | 44/76 |
| C11 | VH1 | Vλ3 | 44/76 NZ98/254 |
| C12 | VH1 | Vλ3 | 44/76 NZ98/254 |
| C13 | VH1 | Vλ6 | 44/76 |
| C14 | VH4 | nd | 44/76 NZ98/254 |
| C15 | VH1 | Vκ3 | 44/76 NZ98/254 |
| C16 | VH3 | Vλ1 | 44/76 NZ98/254 |
| D2 | VH3 | nd | 44/76 |
| D3 | VH3 | Vλ3 | 44/76 |
| D4 | VH3 | Vλ3 | 44/76 |
| D7 | VH4 | Vκ3 | 44/76 |
| D8 | VH3 | Vλ1 | 44/76 |
| D9 | nd | nd | 44/76 |
| D10 | VH4 | Vλ1 | 44/76 |
| D11 | VH4 | nd | 44/76 |
| D12 | VH4 | Vκ3 | 44/76 |
| E1 | VH4 | Vλ1 | 44/76 |
| E2 | VH4 | Vλ6 | 44/76 |
| E3 | VH1 | Vκ1 | 44/76 |
| E4 | VH1 | Vλ8 | 44/76 |
| E5 | VH1 | Vλ8 | 44/76 NZ98/254 |
| E6 | VH1 | Vλ1 | 44/76 |
| E7 | VH1 | Vλ1 | 44/76 NZ98/254 |
| E8 | VH1 | Vλ6 | 44/76 NZ98/254 |
| E9 | VH1 | Vλ6 | 44/76 |
| E10 | VH1 | Vλ6 | 44/76 |
| E11 | VH3 | Vλ2 | 44/76 |

nd = no data.
44/76 = OMV from strain 44/76,
NZ98/254 = OMV from strain NZ98/254

1-16. (canceled)

17. A method for producing and screening an in vitro antibody expression library from a human patient said method comprising the steps of:
a) obtaining one or more populations of antibody producing cells from a human patient, who has been immunochallenged with one or more foreign antigens associated with a particular disease or foreign agent, wherein said cells contain nucleic acid fragments comprising sequences encoding antibody variable domains and are enriched for antibody producing cells which produce antibody molecules directed to said foreign antigens associated with said particular disease or foreign agent;
b) cloning said nucleic acid fragments comprising sequences encoding antibody variable domains, or fragments thereof, from said antibody producing cells into an appropriate prokaryotic expression vector to form a library of said expression vectors capable of expressing a library of antibody molecules;
(c) expressing the library of expression vectors in an appropriate prokaryotic expression system; and
(d) screening the expressed library for antibodies directed to said foreign antigens associated with said particular disease or foreign agent,
wherein said screening step (d) consists of direct screening of said expressed library without prior rounds of selection.

18. (canceled)

19. The method of claim 18 wherein said library is screened against more than one target antigen.

20. The method of claim 19 wherein said target antigens are related.

21. The method of claim 20 wherein cross-reactive antibodies are identified.

22. (canceled)

23. The method of claim 17, wherein step a) of the method is followed by one or more steps wherein the obtained population of antibody producing cells is further enriched for antibody producing cells which produce the desired antibody molecules.

24.-26. (canceled)

27. A method of identifying and/or isolating from an antibody expression library as defined in claim 17 one or more antibody molecules which is a specific binding partner for a target antigen, said method comprising the steps of a) screening an expression library as defined in claim 17 for antibody molecules which bind to a particular target antigen and b) identifying and/or isolating the relevant library member.

28.-42. (canceled)

43. The method of claim 17, wherein said patient has been immunochallenged with said foreign antigen at a time point such that said patient still contains a repertoire of antibody-producing cells which are enriched with cells producing antibodies directed to said foreign antigens associated with said disease or foreign agent, or at a time point such that said patient is still in an active phase of immune response to said foreign antigens associated with said disease or foreign agent.

44. The method of claim 17, wherein said library is obtained from antibody producing cells from a patient obtained up to 40 days after a first or subsequent exposure to said foreign antigen or the outbreak of disease.

45. The method of claim 44, wherein said library is obtained from antibody producing cells from a patient obtained up to 30 days, 20 days, 15 days, 10 days or 7 days after a first or subsequent exposure to said foreign antigen or the outbreak of disease.

46. The method of claim 45, wherein said library is obtained from antibody producing cells from a patient obtained 4 to 10 days or 6 to 7 days after a first or subsequent exposure to said foreign antigen or outbreak of disease.

47. The method of claim 17, wherein said library is obtained from antibody producing cells from a patient obtained 1 to 10 days before said patient shows the highest serum levels of antibodies directed to said foreign antigen associated with said disease or foreign agent.

48. The method of claim 47, wherein said library is obtained from antibody producing cells from a patient obtained 6 to 7 days before the patient shows the highest serum levels of antibodies directed to said foreign antigen associated with said disease or foreign agent.

49. The method of claim 17, wherein said immunochallenge involves a first exposure of said patient to said foreign agent.

50. The method of claim 49, wherein said immunochallenge involves a second or subsequent exposure of said patient to said foreign antigen.

51. The method of claim 17, wherein said immunochallenge involves administration of a vaccine.

52. The method of claim 17, wherein said immunochallenge involves exposure of said patient to an infectious agent.

53. The method of claim 52, wherein said infectious agent is Varicella Zoster virus (VZV), HIV, CMV, hepatitis, herpes, meningococcus or group A Streptococcus.

54. The method of claim 17, wherein said library is obtained from antibody producing cells obtained from a tissue source or other cellular source which is effected by said disease or foreign agent.

55. The method of claim 17, which further comprises identifying or isolating said desired antibodies.

56. The method of claim 17, wherein said prokaryotic expression system is a bacterial expression system.
